(19) United States
(12) Patent Application Publication
(10) Pub. No.: US 2024/0287107 A1
(43) Pub. Date: Aug. 29, 2024

(54) PRODRUGS OF DIMETHYLTRYPTAMINE AND DERIVATIVES THEREOF

(71) Applicant: ATAI Therapeutics, Inc., Encinitas, CA (US)

(72) Inventors: Tanweer A. KHAN, Bridgewater, NJ (US); Robert B. PERNI, Marlborough, MA (US); Alan C. GIBBS, Wyndmoor, PA (US)

(21) Appl. No.: 18/541,172

(22) Filed: May 1, 2024

Related U.S. Application Data
(60) Provisional application No. 63/387,679, filed on Dec. 15, 2022.

(51) Int. Cl. C07F 9/572; A61K 31/675; C07B 59/00; C07F 9/6571

(52) U.S. Cl. CPC C07F 9/5728; A61K 31/675; C07B 59/004; C07F 9/657163; C07B 2200/05

(57) ABSTRACT

Provided herein are compounds of Formula (I-A), Formula (I-B), Formula (II-A), Formula (II-B), pharmaceutically acceptable salts thereof, and compositions thereof, wherein R₁, R₂, R₃, R₇, R₈, R₉, R₁₀, R₁₁, R₁₂, R₁₃, R₁₄, A, B, E, W, X, and n are defined herein. The disclosed compounds are useful for treating various diseases, including depression and associated conditions.

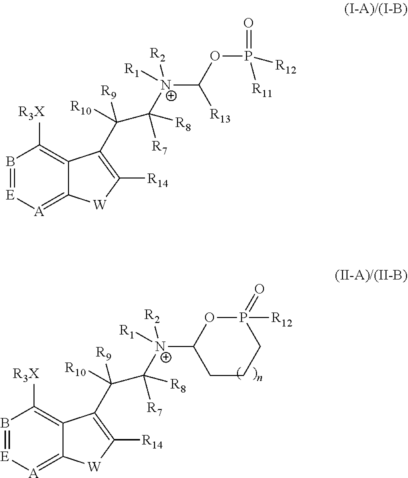

### CROSS-REFERENCE TO RELATED APPLICATIONS

The present application claims priority to U.S. Provisional Patent Application No. 63/387,679 filed Dec. 15, 2022, and which is hereby incorporated by reference in its entirety.

### BACKGROUND OF THE INVENTION

N,N-dimethyltryptamine (DMT) is a naturally occurring compound found in numerous plant species and botanical preparations, such as the hallucinogenic infusion known as ayahuasca, and classified as a classic serotonergic psychedelic that induces intense modifications in perception, emotion, and cognition in humans. At higher doses, DMT has a rapid onset, intense psychedelic effects, and a relatively short duration of action with an estimated half-life of less than fifteen minutes. Like other hallucinogens in the tryptamine family, DMT binds to serotonin receptors to produce euphoria and psychedelic effects. Unfortunately, DMT is metabolically unstable and is readily converted by monoamine oxidases (MAO's) to indoleacetic acid and N-oxidation metabolites resulting in poor oral bioavailability.

Serotonergic psychedelics have also demonstrated promising antidepressant, anxiolytic, and anti-addictive properties.

There remains a need for improved prodrugs of tryptamines, such as N,N-dimethyltryptamine (DMT).

### SUMMARY OF THE INVENTION

In one aspect, the present disclosure provide a compound of Formula (I-A):

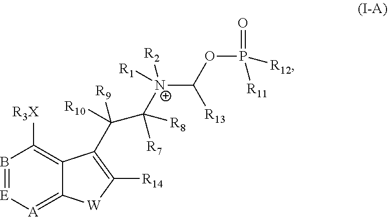

or a pharmaceutically acceptable salt thereof,
wherein:

A is N or C—VR₁₆;

B is N or C—YR₄;

E is N or C—ZR₅, wherein no more than two of A, B, and E are N;

W is O, S, or NR₆;

V, X, Y, and Z are each independently absent, O, S, or NH;

R₁ and R₂ are each independently H, D, deuterated alkyl, optionally substituted alkyl, optionally substituted cycloalkyl, or optionally substituted heteroalkyl;

R₃, R₄, R₅, and R₁₆ are each independently H, D, optionally substituted alkyl, optionally substituted cycloalkyl, optionally substituted heteroalkyl, or —P(O)(OH)₂;

R₆ is H, optionally substituted alkyl, optionally substituted cycloalkyl, alkylene-optionally substituted cycloalkyl, or optionally substituted heteroalkyl;

R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;

R₉ and R₁₀ are each independently H, D, halogen, optionally substituted alkyl, optionally substituted cycloalkyl, or optionally substituted heteroalkyl, or R₉ and R₁₀ taken together with the atom to which they are attached form an oxo;

R₁₁ and R₁₂ are each independently alkyl, deuterated alkyl, —O⁻, —OH, —OD, —O-alkyl, —O-cycloalkyl, —O-alkylene-cycloalkyl, —O-aryl, or —OM, wherein M is a pharmaceutically acceptable cation, or R₁₁ and R₁₂ together with the atoms to which they are attached form a heterocyclyl, and the alkyl, cycloalkyl, aryl and heterocyclyl are each optionally substituted;

R₁₃ is H, D, halogen, deuterated alkyl, optionally substituted alkyl, optionally substituted alkoxy, optionally substituted heteroalkyl, optionally substituted aryl, optionally substituted heteroaryl, alkylene-optionally substituted aryl, —C(O)-(alkylene- optionally substituted aryl), or —O—C(O)—NHR₁₅, or R₁₁ and R₁₃ taken together with the atoms to which they are attached form an optionally substituted heterocyclic ring comprising 4-7 carbon atoms;

R₁₄ is H, D, halogen, haloalkyl, —OH, an optionally substituted alkyl, optionally substituted cycloalkyl, or optionally substituted heteroalkyl; and

R₁₅ is H, optionally substituted alkyl, optionally substituted cycloalkyl, optionally substituted aryl, or optionally substituted heteroaryl.

In one aspect, the present disclosure provides a compound of Formula (I-B):

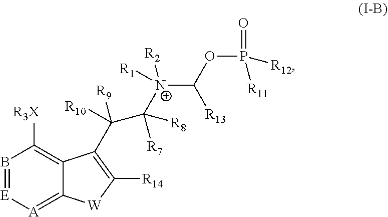

or a pharmaceutically acceptable salt thereof,
wherein:

A is N or CH;

B is N or C—YR₄;

E is N or C—ZR₅, wherein no more than two of A, B, and E are N;

W is O, S, or NR₆;

X, Y, and Z are each independently absent, O, S, or NH;

R₁ and R₂ are each independently H, D, optionally substituted alkyl, optionally substituted cycloalkyl, or optionally substituted heteroalkyl;

R₃, R₄, and R₅ are each independently H, D, optionally substituted alkyl, optionally substituted cycloalkyl, optionally substituted heteroalkyl, or —P(O)(OH)₂;

R₆ is H, optionally substituted alkyl, optionally substituted cycloalkyl, alkylene-optionally substituted cycloalkyl, or optionally substituted heteroalkyl;

R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;

R₉ and R₁₀ are each independently H, D, halogen, optionally substituted alkyl, optionally substituted cycloalkyl, or optionally substituted heteroalkyl, or R₉ and R₁₀ taken together with the atom to which they are attached form an oxo;

R₁₁ and R₁₂ are each independently O⁻, —OH, —OD, —O-alkyl, —O-cycloalkyl, —O— alkylene-cycloalkyl, —O-aryl, or —OM, wherein M is a pharmaceutically acceptable cation, or R₁₁ and R₁₂ together with the atoms to which they are attached form a heterocyclyl, and the alkyl, cycloalkyl, aryl and heterocyclyl are each optionally substituted;

R₁₃ is H, D, halogen, optionally substituted alkyl, optionally substituted alkoxy, optionally substituted heteroalkyl, optionally substituted aryl, optionally substituted heteroaryl, alkylene-optionally substituted aryl, —C(O)-(alkylene- optionally substituted aryl), or —O—C(O)—NHR₁₅, or R₁₁ and R₁₃ taken together with the atoms to which they are attached form an optionally substituted heterocyclic ring comprising 4-7 carbon atoms;

R₁₄ is H, D, halogen, —OH, an optionally substituted alkyl, optionally substituted cycloalkyl, or optionally substituted heteroalkyl; and

R₁₅ is H, optionally substituted alkyl, optionally substituted cycloalkyl, optionally substituted aryl, or optionally substituted heteroaryl.

In another aspect, the present disclosure provides a compound of Formula (II-A): compound of Formula (II-A):

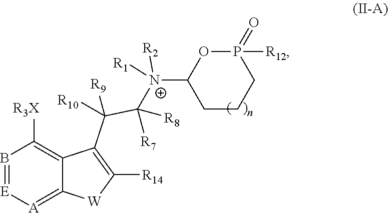

or a pharmaceutically acceptable salt thereof,
wherein:

A is N or C—VR₁₆;

B is N or C—YR₄;

E is N or C—ZR₅, wherein no more than two of A, B, and E are N;

W is O, S, or NR₆;

V, X, Y, and Z are each independently absent, O, S, or NH;

R₁ and R₂ are each independently H, D, deuterated alkyl, optionally substituted alkyl, optionally substituted cycloalkyl, or optionally substituted heteroalkyl;

R₃, R₄, R₅, and R₁₆ are each independently H, D, optionally substituted alkyl, optionally substituted cycloalkyl, optionally substituted heteroalkyl, or —P(O)(OH)₂;

R₆ is H, optionally substituted alkyl, optionally substituted cycloalkyl, optionally substituted alkylene-cycloalkyl, or optionally substituted heteroalkyl;

R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;

R₉ and R₁₀ are each independently H, D, halogen, —OH, optionally substituted alkyl, optionally substituted cycloalkyl, or optionally substituted heteroalkyl, or R₉ and R₁₀ taken together with the atom to which they are attached form an oxo;

R₁₂ is —O⁻, —OH, —OD, —O-alkyl, —O-cycloalkyl, —O-alkylene-cycloalkyl, —O-aryl, or —OM, wherein M is a pharmaceutically acceptable cation, and the alkyl, cycloalkyl, and aryl are each optionally substituted;

R₁₄ is H, D, halogen, —OH, optionally substituted alkyl, optionally substituted cycloalkyl, or optionally substituted heteroalkyl; and

n is 0, 1, or 2.

In another aspect, the present disclosure provides a compound of Formula (II-B):

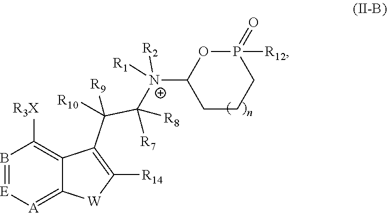

or a pharmaceutically acceptable salt thereof,
wherein:

A is N or CH;

B is N or C—YR₄;

E is N or C—ZR₅, wherein no more than two of A, B, and E are N;

W is O, S, or NR₆;

X, Y, and Z are each independently absent, O, S, or NH;

R₁ and R₂ are each independently H, D, optionally substituted alkyl, optionally substituted cycloalkyl, or optionally substituted heteroalkyl;

R₃, R₄, and R₅ are each independently H, D, optionally substituted alkyl, optionally substituted cycloalkyl, optionally substituted heteroalkyl, or —P(O)(OH)₂;

R₆ is H, optionally substituted alkyl, optionally substituted cycloalkyl, optionally substituted alkylene-cycloalkyl, or optionally substituted heteroalkyl;

R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;

R₉ and R₁₀ are each independently H, D, halogen, —OH, optionally substituted alkyl, optionally substituted cycloalkyl, or optionally substituted heteroalkyl, or R₉ and R₁₀ taken together with the atom to which they are attached form an oxo;

R₁₂ is O⁻, —OH, —OD, —O-alkyl, —O-cycloalkyl, —O-alkylene-cycloalkyl, —O-aryl, or —OM, wherein M is a pharmaceutically acceptable cation, and the alkyl, cycloalkyl, and aryl are optionally substituted;

R₁₄ is H, D, halogen, —OH, optionally substituted alkyl, optionally substituted cycloalkyl, or optionally substituted heteroalkyl; and

n is 0, 1, or 2.

In embodiments, the present disclosure provides a pharmaceutical composition comprising a compound of Formula (I-A) or a pharmaceutically acceptable salt thereof and a pharmaceutically acceptable excipient.

In embodiments, the present disclosure provides a pharmaceutical composition comprising a compound of Formula (I-B) or a pharmaceutically acceptable salt thereof and a pharmaceutically acceptable excipient.

In embodiments, the present disclosure provides a pharmaceutical composition comprising a compound of Formula (II-A) or a pharmaceutically acceptable salt thereof and a pharmaceutically acceptable excipient.

In embodiments, the present disclosure provides a pharmaceutical composition comprising a compound of Formula (II-B) or a pharmaceutically acceptable salt thereof and a pharmaceutically acceptable excipient.

### BRIEF DESCRIPTION OF THE DRAWINGS

FIGS. 1A-1C show PK studies of N-phosphonooxymethyl prodrug Compound 1 (VLS-02-23-0; 6-1-2) and metabolite (VLS-02-023-10) following intravenous (1 mg/kg) and oral administration (30 mg/kg) to male CD1 mice (in plasma).

FIGS. 2A-2B show PK studies of N-phosphonooxymethyl prodrug Compound 1 (VLS-02-23-0; 6-1-2) and metabolite (VLS-02-023-10) following intravenous (1 mg/kg) and oral administration (30 mg/kg) to male CD1 mice (in the brain).

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “treating” as used herein with regard to a patient, refers to improving at least one symptom of the patient's disorder. In embodiments, treating can be improving, or at least partially ameliorating a disorder or one or more symptoms of a disorder.

The term “preventing” as used herein with regard to a patient or subject, refers to preventing the onset of disease development if none had occurred, preventing the disease or disorder from occurring in a subject or a patient that may be predisposed to the disorder or disease but has not yet been diagnosed as having the disorder or disease, and/or preventing further disease/disorder development if already present.

The term “therapeutically effective” applied to dose or amount refers to that quantity of a compound or pharmaceutical formulation that is sufficient to result in a desired clinical benefit after administration to a patient in need thereof.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C_{3_4}, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the groups described herein (e.g., alkyl, alkenyl, alkynyl, alkoxy, aryl, cycloalkyl, cycloalkenyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

### Compounds

The present disclosure provides compounds that are prodrugs of tryptamines and related scaffolds, for example N,N-dimethyltryptamine (DMT) as well as pharmaceutical compositions thereof.

DMT is metabolically unstable and is readily converted by monoamine oxidases (MAO's) to indoleacetic acid and N-oxidation metabolites resulting in poor oral bioavailability. In embodiments, the compounds of the present disclosure allow for the controlled release of DMT. Advantages of compounds of the present disclosure may include increased metabolic stability, increased absorption, decreased maximal plasma concentrations of parent drug DMT over time, and less frequent dosing. In embodiments, compounds of the present disclosure prevent or inhibit N-oxidation to promote oral bioavailability and increased exposure.

In embodiments, the DMT compounds of the present disclosure comprise a phosphate moiety, a phosphonate moiety, or a derivate thereof. Without being bound by theory such compounds may for example, increase bioavailability by virtue of active transport of the prodrug in the small intestine by high-capacity nutrient transporters, including mono-carboxylate transporter-1 (MCT-1). The DMT prodrugs disclosed herein may also reduce abuse potential by preventing absorption by snorting or insufflation.

### Compounds of Formulas (I-A) and (II-A)

In embodiments, the present disclosure provides a compound of Formula (I-A):

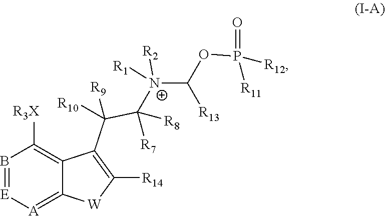

or a pharmaceutically acceptable salt thereof,
wherein:

### A is N or C—VR₁₆;

### B is N or C—YR₄;

E is N or C—ZR₅, wherein no more than two of A, B, and E are N;

### W is O, S, or NR₆;

V, X, Y, and Z are each independently absent, O, S, or NH;
R₁ and R₂ are each independently H, D, deuterated alkyl, optionally substituted alkyl, optionally substituted cycloalkyl, or optionally substituted heteroalkyl;
R₃, R₄, R₅, and R₁₆ are each independently H, D, optionally substituted alkyl, optionally substituted cycloalkyl, optionally substituted heteroalkyl, or —P(O)(OH)₂;
R₆ is H, optionally substituted alkyl, optionally substituted cycloalkyl, alkylene- optionally substituted cycloalkyl, or optionally substituted heteroalkyl;
R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;
R₉ and R₁₀ are each independently H, D, halogen, optionally substituted alkyl, optionally substituted cycloalkyl, or optionally substituted heteroalkyl, or R₉ and R₁₀ taken together with the atom to which they are attached form an oxo;
R₁₁ and R₁₂ are each independently alkyl, deuterated alkyl, O⁻, —OH, —OD, —O-alkyl, —O— cycloalkyl, —O-alkylene-cycloalkyl, —O-aryl, or —OM, wherein M is a pharmaceutically acceptable cation, or R₁₁ and R₁₂ together with the atoms to which they are attached form a heterocyclyl, and the alkyl, cycloalkyl, aryl and heterocyclyl are each optionally substituted;
R₁₃ is H, D, halogen, deuterated alkyl, optionally substituted alkyl, optionally substituted alkoxy, optionally substituted heteroalkyl, optionally substituted aryl, optionally substituted heteroaryl, alkylene-optionally substituted aryl, —C(O)-(alkylene- optionally substituted aryl), or -G-C(O)—NHR₁₅, or R₁₁ and R₁₃ taken together with the atoms to which they are attached form an optionally substituted heterocyclic ring comprising 4-7 carbon atoms;
R₁₄ is H, D, halogen, haloalkyl, —OH, an optionally substituted alkyl, optionally substituted cycloalkyl, or optionally substituted heteroalkyl; and
R₁₅ is H, optionally substituted alkyl, optionally substituted cycloalkyl, optionally substituted aryl, or optionally substituted heteroaryl.

In embodiments, the present disclosure provides a compound of Formula (II-A):

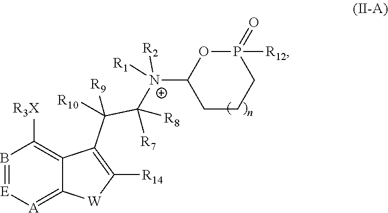

or a pharmaceutically acceptable salt thereof,
wherein:

### A is N or C—VR₁₆;

### B is N or C—YR₄;

E is N or C—ZR₅, wherein no more than two of A, B, and E are N;

### W is O, S, or NR₆;

V, X, Y, and Z are each independently absent, O, S, or NH;
R₁ and R₂ are each independently H, D, deuterated alkyl, optionally substituted alkyl, optionally substituted cycloalkyl, or optionally substituted heteroalkyl;
R₃, R₄, R₅, and R₁₅ are each independently H, D, optionally substituted alkyl, optionally substituted cycloalkyl, optionally substituted heteroalkyl, or —P(O)(OH)₂;
R₆ is H, optionally substituted alkyl, optionally substituted cycloalkyl, optionally substituted alkylene-cycloalkyl, or optionally substituted heteroalkyl;
R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;
R₉ and R₁₀ are each independently H, D, halogen, —OH, optionally substituted alkyl, optionally substituted cycloalkyl, or optionally substituted heteroalkyl, or R₉ and R₁₀ taken together with the atom to which they are attached form an oxo;
R₁₂ is O⁻, —OH, —OD, —O-alkyl, —O-cycloalkyl, —O-alkylene-cycloalkyl, —O-aryl, or —OM, wherein M is a pharmaceutically acceptable cation, and the alkyl, cycloalkyl, and aryl are optionally substituted;
R₁₄ is H, D, halogen, —OH, optionally substituted alkyl, optionally substituted cycloalkyl, or optionally substituted heteroalkyl; and
n is 0, 1, or 2.

In embodiments, A is N. In embodiments, A is C—VR₁₆.

In embodiments, B is N. In embodiments, B is C—YR₄.

In embodiments, E is N. In embodiments, E is C—ZR₅.

In embodiments, W is NR₆ or O. In embodiments, W is NR₆ or S. In embodiments, W is O or S. In embodiments, W is NR₆. In embodiments, W is O. In embodiments, W is S.

In embodiments, V, X, Y, and Z are each independently absent, O, S, or N(C₁₋₅)alkyl. In embodiments, V, X, Y, and Z are each independently absent or O. In embodiments, V, X, Y, and Z are each independently absent or NH. In embodiments, V, X, Y, and Z are each independently absent or S. In embodiments, V, X, Y, and Z are each absent.

In embodiments, X is O, and V, Y and Z are absent. In embodiments, Y is G, and V, X and Z are absent. In embodiments, Z is O, and V, X and Y are absent. In embodiments, V is G, and X, Y, and Z are absent. In embodiments, X is S, and V, Y and Z are absent. In embodiments, Y is S, and V, X and Z are absent. In embodiments, Z is S, and V, X and Y are absent. In embodiments, V is S, and X, Y, and Z are absent. In embodiments, X is NH, and V, Y and Z are absent. In embodiments, Y is NH, and V, X and Z are absent. In embodiments, Z is NH, and V, X and Y are absent. In embodiments, V is NH, and X, Y, and Z are absent. In embodiments, X is N(C₁₋₅)alkyl, and V, Y and Z are absent. In embodiments, Y is N(C₁₋₅)alkyl, and V, X and Z are absent. In embodiments, Z is N(C₁₋₅)alkyl, and V, X and Y are absent. In embodiments, V is N(C₁₋₅)alkyl, and X, Y, and Z are absent.

In embodiments, R₁ and R₂ are each independently H, optionally substituted alkyl, optionally substituted cycloalkyl, or optionally substituted heteroalkyl. In embodiments, R₁ and R₂ are each independently H, optionally substituted C₁₋₅alkyl, optionally substituted C₃₋₆cycloalkyl, or optionally substituted C₁₋₅cycloalkyl heteroalkyl. In some embodiments, R₁ and R₂ are each independently H or alkyl. In some embodiments, R₁ and R₂ are each independently alkyl or deuterated alkyl. In some embodiments, R₁ and R₂ are each independently H or deuterated alkyl. In embodiments, R₁ and R₂ are each independently H or C₁₋₅alkyl. In embodiments, R₁ and R₂ are each independently H or deuterated C₁₋₅alkyl. In embodiments, R₁ and R₂ are each independently alkyl or deuterated C₁₋₅alkyl. In embodiments, R₁ and R₂ are each alkyl. In embodiments, R₁ and R₂ are each deuterated alkyl. In embodiments, R₁ and R₂ are each C₁₋₅alkyl. In embodiments, R₁ and R₂ are each deuterated C₁₋₅alkyl. In embodiments, R₁ and R₂ are each independently methyl, ethyl, or isopropyl. In embodiments, R₁ and R₂ are each independently deuterated methyl, deuterated ethyl, or deuterated isopropyl. In embodiments, R₁ and R₂ are each methyl. In embodiments, R₁ and R₂ are each deuterated methyl. In embodiments, R₁ and R₂ taken together with the nitrogen to which they are attached form a 4- to 6-membered heterocyclyl, e.g., azetidine, pyrrolidine, or piperidine.

In embodiments, R₃, R₄, R₅, and R₁₆ are each independently H, D, optionally substituted C₁₋₅alkyl, optionally substituted C₃₋₆cycloalkyl, optionally substituted C₁₋₅heteroalkyl, —P(O)(OC₁₋₅alkyl)₂, —P(O)(OPh)₂, or —P(O)(OH)₂. In embodiments, R₃, R₄, R₅, and R₁₆ are each independently H, optionally substituted C₁₋₅alkyl, optionally substituted C₃₋₆cycloalkyl, optionally substituted C₁₋₅heteroalkyl, —P(O)(OC₁₋₅alkyl)₂, —P(O)(OPh)₂, or —P(O)(OH)₂. In embodiments, R₃, R₄, R₅, and R₁₆ are each independently H, optionally substituted C₁₋₅alkyl, optionally substituted C₃₋₆cycloalkyl, —P(O)(OC₁₋₅alkyl)₂, —P(O)(OPh)₂, or —P(O)(OH)₂. In embodiments, R₃, R₄, R₅, and R₁₆ are each independently H, optionally substituted C₁₋₅alkyl, optionally substituted C₃₋₆cycloalkyl, or —P(O)(OH)₂. In embodiments, R₃, R₄, R₅, and R₁₆ are each independently H, optionally substituted C₁₋₅alkyl, or —P(O)(OH)₂. In embodiments, the C₁₋₅alkyl is methyl, ethyl, propyl, isopropyl, or t-butyl. In embodiments, the C₁₋₅alkyl is methyl, ethyl, or isopropyl. In embodiments, R₃, R₄, R₅, and R₁₆ are each H.

In embodiments, R₃ is optionally substituted C₁₋₅alkyl or —P(O)(OH)₂, and R₄, R₅, and R₁₆ are each H. In embodiments, R₄ is optionally substituted C₁₋₅alkyl or —P(O)(OH)₂, and R₃, R₅, and R₁₆ are each H. In embodiments, R₅ is optionally substituted C₁₋₅alkyl or —P(O)(OH)₂, and R₃, R₄, and R₁₆ are each H. In embodiments, R₁₆ is optionally substituted C₁₋₅alkyl or —P(O)(OH)₂, and R₃, R₄, and R₅ are each H. In embodiments, the C₁₋₅alkyl is methyl, ethyl, propyl, isopropyl, or t-butyl. In embodiments, the C₁₋₅alkyl is methyl, ethyl, or isopropyl.

In embodiments, X is absent, and R₃ is H, halogen, optionally substituted C₁₋₅alkyl, optionally substituted C₃₋₆cycloalkyl, or —P(O)(OH)₂. In embodiments, Y is absent, and R₄ is H, halogen, optionally substituted C₁₋₅alkyl, optionally substituted C₃₋₆cycloalkyl, or —P(O)(OH)₂. In embodiments, Z is absent, and R₃ is H, halogen, optionally substituted C₁₋₅alkyl, optionally substituted C₃₋₆cycloalkyl, or —P(O)(OH)₂. In embodiments, V is absent, and R₁₆ is H, halogen, optionally substituted C₁₋₅alkyl, optionally substituted C₃₋₆cycloalkyl, or —P(O)(OH)₂. In embodiments, the C₁₋₅alkyl is methyl, ethyl, propyl, isopropyl, or t-butyl. In embodiments, the C₁₋₅alkyl is methyl, ethyl, or isopropyl. In embodiments, the halogen is F, Cl, or Br.

In embodiments, R₆ is H, optionally substituted alkyl, optionally substituted cycloalkyl, optionally substituted alkylene-cycloalkyl, optionally substituted alkylene-heterocyclyl, or optionally substituted alkylene-aryl. In embodiments, R₆ is H, optionally substituted alkyl, optionally substituted cycloalkyl, or optionally substituted alkylene-cycloalkyl. In embodiments, the optionally substituted alkyl is an optionally substituted C₁₋₅alkyl. In embodiments, the optionally substituted cycloalkyl is an optionally substituted C₃₋₆cycloalkyl. In embodiments, the optionally substituted aryl is an optionally substituted phenyl. In embodiments, the optionally substituted heterocyclyl is an optionally substituted 4- to 6-membered heterocyclyl, e.g., an azetidine, pyrrolidine, piperidine, morpholine, thiomorpholine, piperazine, each of which is optionally substituted. In embodiments, the optionally substituted alkylene is an optionally substituted C₁₋₃alkylene. In embodiments, the optionally substituted alkylene is an optionally substituted methylene. In embodiments, R₆ is H, C₁₋₅alkyl, C₃₋₆cycloalkyl or C(═O)(C₁₋₅ alkyl). In embodiments, R₆ is H, C₁₋₅alkyl, or C₃₋₈ cycloalkyl. In embodiments, R₆ is H or C₁₋₅alkyl. In embodiments, R₆ is H. In embodiments, R₆ is C₁₋₅alkyl. In embodiments, the C₁₋₅alkyl is methyl, ethyl, propyl, isopropyl, or t-butyl. In embodiments, the optionally substituted C₁₋₅alkyl is —C(O)—C₁₋₄alkyl, —C(O)—N(H)(C₁₋₄alkyl), or —C(O)—OC₁₋₄alkyl.

In embodiments, R₇ and R₈ are each independently H, halogen, optionally substituted alkyl, —OH, optionally substituted —O-alkyl or optionally substituted —O— cycloalkyl. In embodiments, R₇ and R₈ are each independently H, halogen, optionally substituted C₁₋₅alkyl, —OH, optionally substituted —OC₁₋₅alkyl, or optionally substituted —O—C₃₋₆cycloalkyl. In embodiments, R₇ and R₈ are each independently H or optionally substituted C₁₋₅alkyl. In embodiments, R₇ and R₈ are each H. In embodiments, R₇ and R₈ are each optionally substituted C₁₋₅alkyl. In embodiments, R₇ and R₈ are each halogen. In embodiments, the halogen is F. In embodiments, the C₁₋₅alkyl is methyl, ethyl, or isopropyl. In embodiments, the C₁₋₅alkyl is methyl. In embodiments, the C₁₋₅alkyl is ethyl.

In embodiments, R₇ and R₈ taken together with the atom to which they are attached form an oxo or an optionally substituted cycloalkyl ring. In embodiments, R₇ and R₈ taken together with the atom to which they are attached form an oxo. In embodiments, R₇ and R₈ taken together with the atom to which they are attached form an optionally substituted cycloalkyl ring. In embodiments, the optionally substituted cycloalkyl ring is an optionally substituted C₃₋₆cycloalkyl ring. In embodiments, the optionally substituted cycloalkyl ring is an optionally substituted cyclopropyl. In embodiments, R₇ is H and R₅ is optionally substituted C₁₋₅alkyl. In embodiments, R₇ is H and R₅ is F. In embodiments, R₇ is H and R₅ is —OH. In embodiments, R₇ is H and R₅ is —OCH₃. In embodiments, R₇ is F and R₅ is F. In embodiments, R₇ and R₈ are each optionally substituted C₁₋₅alkyl. In embodiments, the C₁₋₅alkyl is methyl.

In embodiments, R₉ and R₁₀ are each independently H, halogen, optionally substituted alkyl, —OH, optionally substituted —O-alkyl or optionally substituted —O— cycloalkyl. In embodiments, R₉ and R₁₀ are each independently H, halogen, optionally substituted C₁₋₅alkyl, —OH, optionally substituted —OC₁₋₅alkyl, or optionally substituted —O—C₃₋₆cycloalkyl. In embodiments, R₉ and R₁₀ are each independently H or optionally substituted C₁₋₅alkyl. In embodiments, R₉ and R₁₀ are each H. In embodiments, R₉ and R₁₀ are each optionally substituted C₁₋₅alkyl. In embodiments, R₉ and R₁₀ are each halogen. In embodiments, the halogen is F. In embodiments, the C₁₋₅alkyl is methyl, ethyl, or isopropyl. In embodiments, the C₁₋₅alkyl is methyl. In embodiments, the C₁₋₅alkyl is ethyl.

In embodiments, R₉ and R₁₀ taken together with the atom to which they are attached form an optionally substituted cycloalkyl ring. In embodiments, the optionally substituted cycloalkyl ring is an optionally substituted C₃₋₆cycloalkyl ring. In embodiments, the optionally substituted cycloalkyl ring is an optionally substituted cyclopropyl. In embodiments, R₉ is H and R₁₀ is optionally substituted C₁₋₅alkyl. In embodiments, R₉ is H and R₁₀ is F. In embodiments, R₉ is H and R₁₀ is —OH. In embodiments, R₉ is H and R₁₀ is —OCH₃. In embodiments, R₉ and R₁₀ are each F. In embodiments, R₉ and R₁₀ are each optionally substituted C₁₋₅alkyl. In embodiments, the C₁₋₅alkyl is methyl.

In embodiments of Formula (I-A), R₁₁ and R₁₂ are each independently alkyl, deuterated alkyl, O⁻, —OH, optionally substituted —O-alkyl, optionally substituted —O-cycloalkyl, optionally substituted —O-alkylene-cycloalkyl, optionally substituted —O-aryl, or —OM, wherein M is a pharmaceutically acceptable cation. In embodiments, the optionally substituted —O-alkyl is an optionally substituted —O-haloalkyl, a hemiaminal, an acetal, or an amino acid residue. In embodiments, R₁₁ and R₁₂ are each independently O⁻, —OH, optionally substituted —O—C₁₋₅alkyl, optionally substituted —O—C₁₋₅haloalkyl, optionally substituted —O—C₃₋₆cycloalkyl, or -OM. In embodiments, R₁₁ and R₁₂ are each independently O⁻, —OH, optionally substituted —O—C₁₋₅alkyl, optionally substituted —O—C₁₋₅haloalkyl, or -OM. In embodiments, R₁₁ is O⁻ and R₁₂ is —OH, optionally substituted —O—C₁₋₅alkyl, optionally substituted —O—C₁₋₅haloalkyl, or -OM. In embodiments, R₁₁ and R₁₂ are each optionally substituted —O—C₁₋₅alkyl. In embodiments, R₁₁ and R₁₂ are each optionally substituted —O—C₁₋₃alkyl. In embodiments, R₁₁ and R₁₂ are each independently —OMe, —OEt, —OiPr, or -OtBu. In embodiments, R₁₁ and R₁₂ are each optionally substituted —O—C₁₋₅haloalkyl. In embodiments, the —O—C₁₋₅haloalkyl is —OCH₃, —OCH₂F, —OCHF₂, or —OCH₂CF₃. In embodiments, the —O—C₁₋₅haloalkyl is a —OCH₂CF₃. In embodiments, R₁₁ and R₁₂ are each independently alkyl, deuterated alkyl, O⁻, —OH, —OD, —O-alkyl, or —O— cycloalkyl. In embodiments, R₁₁ and R₁₂ are each independently alkyl or deuterated alkyl. In embodiments, M is Na⁺, K⁺, NH₄⁺, or Ca⁺². In embodiments, M is Na⁺, K⁺, or NH₄⁺.

In embodiments of Formula (I-A), R₁₁ and R₁₂ taken together with the atoms to which they are attached form a heterocyclyl. In embodiments, R₁₁ and R₁₂ taken together with the atoms to which they are attached form a 5- to 12-membered heterocyclyl. In embodiments, the heterocyclyl has the structure:

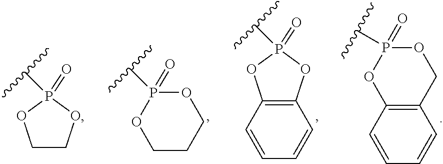

In embodiments of Formula (II-A), R₁₂ is O⁻, —OH, optionally substituted —O-alkyl, optionally substituted —O-cycloalkyl, optionally substituted —O-alkylene-cycloalkyl, optionally substituted —O-aryl, or —OM, wherein M is a pharmaceutically acceptable cation. In embodiments, the optionally substituted —O-alkyl is an optionally substituted —O-haloalkyl, a hemiaminal, an acetal, or an amino acid residue. In embodiments, R₁₂ is O⁻, —OH, —O-alkyl, —O-haloalkyl, —O-cycloalkyl, or -OM. In embodiments, R₁₂ is O⁻, —OH, optionally substituted —O-alkyl, optionally substituted —O-haloalkyl, or -OM. In embodiments, R₁₂ is O⁻, optionally substituted —O-alkyl, or optionally substituted —O-haloalkyl. In embodiments, R₁₂ is optionally substituted —O-alkyl. In embodiments, the optionally substituted —O-alkyl is an optionally substituted —O—C₁₋₅alkyl. In embodiments, the —O-alkyl is —OMe, —OEt, —OiPr, or -OtBu. In embodiments, R₁₂ is —O-haloalkyl. In embodiments, the optionally substituted —O-haloalkyl is optionally substituted —O—C₁₋₅haloalkyl. In some embodiments, the —O-haloalkyl is —OCH₂CF₃. In embodiments, R₁₂ is O⁻, —OH, —O—C₁₋₅alkyl, —O—C₁₋₅haloalkyl, —O—C₃₋₆cycloalkyl, or -OM. In embodiments, M is Na⁺, K⁺, NH₄⁺, or Ca⁺². In embodiments, M is Na⁺, K⁺, or NH₄⁺. In embodiments, the —O-alkyl is —OMe, —OEt, —OiPr, or -OtBu. In embodiments, the —O—C₁₋₅haloalkyl is —OCH₃, —OCH₂F, —OCHF₂, or —OCH₂CF₃. In embodiments, the —O—C₁₋₅haloalkyl is —OCH₂CF₃.

In embodiments, R₁₃ is H, D, or C₁₋₅alkyl. In embodiments, R₁₃ is H or —C₁₋₅alkyl. In embodiments, the C₁₋₅alkyl is methyl, ethyl, propyl, isopropyl, or butyl. In embodiments, R₁₃ is H, methyl, ethyl, or isopropyl.

In embodiments of Formula (I), R₁₁ and R₁₃ taken together with the atoms to which they are attached form an optionally substituted heterocyclic ring comprising 4-7 carbon atoms. In embodiments, R₁ and R₁₃ taken together with the atoms to which they are attached form an optionally substituted 5-to 7-membered heterocyclic ring. In embodiments, R₁₁ and R₁₃ taken together with the atoms to which they are attached form an optionally substituted 5- or 6-membered heterocyclic ring.

In embodiments, R₁₄ is H, halogen, —OH, optionally substituted alkyl, optionally substituted cycloalkyl, or optionally substituted heteroalkyl. In embodiments, R₁₄ is H, halogen, or optionally substituted alkyl. In embodiments, R₁₄ is H or alkyl. In some embodiments, R₁₄ is H. In embodiments, R₁₄ is alkyl. In embodiments, the alkyl is a C₁-C₅alkyl. In embodiments, the C₁-C₅ alkyl is s methyl, ethyl, or isopropyl. In embodiments, R₁₄ is halogen. In embodiments, the halogen is Cl, Br, or F. In embodiments, the cycloalkyl is a C₃-C₆ cycloalkyl. In embodiments, the cycloalkyl is cyclopropyl. In embodiments, the optionally substituted heteroalkyl is —CH₂—O—C₁₋₅alkyl. In embodiments, the optionally substituted heteroalkyl is —CH₂—N—(H)(C₁₋₅alkyl) or —CH₂—N—(C₁₋₅alkyl)₂.

### Compounds of Formulas (I-B) and (II-B)

In embodiments, the present disclosure provides a compound of Formula (I-B):

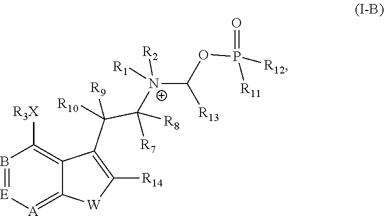

or a pharmaceutically acceptable salt thereof,
wherein:

### A is N or CH;

### B is N or C—YR₄;

E is N or C—ZR₅, wherein no more than two of A, B, and E are N;

### W is O, S, or NR₆;

X, Y, and Z are each independently absent, O, S, or NH;
R₁ and R₂ are each independently H, D, optionally substituted alkyl, optionally substituted cycloalkyl, or optionally substituted heteroalkyl;
R₃, R₄, and R₅ are each independently H, D, optionally substituted alkyl, optionally substituted cycloalkyl, optionally substituted heteroalkyl, or —P(O)(OH)₂;
R₆ is H, optionally substituted alkyl, optionally substituted cycloalkyl, alkylene- optionally substituted cycloalkyl, or optionally substituted heteroalkyl;
R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;
R₉ and R₁₀ are each independently H, D, halogen, optionally substituted alkyl, optionally substituted cycloalkyl, or optionally substituted heteroalkyl, or R₉ and R₁₀ taken together with the atom to which they are attached form an oxo;
R₁₁ and R₁₂ are each independently O⁻, —OH, —OD, —O-alkyl, —O-cycloalkyl, —O-alkylene-cycloalkyl, —O-aryl, or —OM, wherein M is a pharmaceutically acceptable cation, or R₁₁ and R₁₂ together with the atoms to which they are attached form a heterocyclyl, and the alkyl, cycloalkyl, aryl and heterocyclyl are each optionally substituted;
R₁₃ is H, D, halogen, optionally substituted alkyl, optionally substituted alkoxy, optionally substituted heteroalkyl, optionally substituted aryl, optionally substituted heteroaryl, alkylene-optionally substituted aryl, —C(O)-(alkylene- optionally substituted aryl), or —O—C(O)—NHR₁₅, or R₁₁ and R₁₃ taken together with the atoms to which they are attached form an optionally substituted heterocyclic ring comprising 4-7 carbon atoms;
R₁₄ is H, D, halogen, —OH, an optionally substituted alkyl, optionally substituted cycloalkyl, or optionally substituted heteroalkyl; and
R₁₅ is H, optionally substituted alkyl, optionally substituted cycloalkyl, optionally substituted aryl, or optionally substituted heteroaryl.

In embodiments, is the present disclosure provides a compound of Formula (II-B):

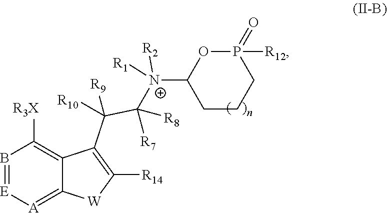

or a pharmaceutically acceptable salt thereof,
wherein:

### A is N or CH;

### B is N or C—YR₄;

E is N or C—ZR₅, wherein no more than two of A, B, and E are N;

### W is O, S, or NR₆;

X, Y, and Z are each independently absent, O, S, or NH;
R₁ and R₂ are each independently H, D, optionally substituted alkyl, optionally substituted cycloalkyl, or optionally substituted heteroalkyl;
R₃, R₄, and R₅ are each independently H, D, optionally substituted alkyl, optionally substituted cycloalkyl, optionally substituted heteroalkyl, or —P(O)(OH)₂;
R₆ is H, optionally substituted alkyl, optionally substituted cycloalkyl, optionally substituted alkylene-cycloalkyl, or optionally substituted heteroalkyl;
R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;
R₉ and R₁₀ are each independently H, D, halogen, —OH, optionally substituted alkyl, optionally substituted cycloalkyl, or optionally substituted heteroalkyl, or R₉ and R₁₀ taken together with the atom to which they are attached form an oxo;
R₁₂ is O⁻, —OH, —OD, —O-alkyl, —O-cycloalkyl, —O-alkylene-cycloalkyl, —O-aryl, or —OM, wherein M is a pharmaceutically acceptable cation, and the alkyl, cycloalkyl, and aryl are optionally substituted;
R₁₄ is H, D, halogen, —OH, optionally substituted alkyl, optionally substituted cycloalkyl, or optionally substituted heteroalkyl; and
n is 0, 1, or 2.

In embodiments, A is N. In embodiments, A is CH.

In embodiments, B is N. In embodiments, B is C—YR₄.

In embodiments, E is N. In embodiments, E is C—ZR₅.

In embodiments of Formula (I), W is NR₆ or O. In embodiments, W is NR₆ or S. In embodiments, W is O or S. In embodiments, W is NR₆. In embodiments, W is O. In embodiments, W is S.

In embodiments, X, Y, and Z are each independently absent, O, S, or N(C₁₋₅)alkyl. In embodiments, X, Y, and Z are each independently absent or O. In embodiments, X, Y, and Z are each independently absent or NH. In embodiments, X, Y, and Z are each independently absent or S. In embodiments, X, Y, and Z are each absent.

In embodiments, X is O, and Y and Z are absent. In embodiments, Y is G, and X and Z are absent. In embodiments, Z is O, and X and Y are absent. In embodiments, X is S, and Y and Z are absent. In embodiments, Y is S, and X and Z are absent. In embodiments, Z is S, and X and Y are absent. In embodiments, X is NH, and Y and Z are absent. In embodiments, Y is NH, and X and Z are absent. In embodiments, Z is NH, and X and Y are absent. In embodiments, X is N(C₁₋₅)alkyl, and Y and Z are absent. In embodiments, Y is N(C₁₋₅)alkyl, and X and Z are absent. In embodiments, Z is N(C₁₋₅)alkyl, and X and Y are absent.

In embodiments, R₁ and R₂ are each independently H, optionally substituted alkyl, optionally substituted cycloalkyl, or optionally substituted heteroalkyl. In embodiments, R₁ and R₂ are each independently H, optionally substituted C₁₋₅alkyl, optionally substituted C₃₋₆Cycloalkyl, or optionally substituted C₁₋₅cycloalkyl heteroalkyl. In some embodiments, R₁ and R₂ are each independently H or alkyl. In embodiments, R₁ and R₂ are each independently H or C₁₋₅alkyl. In embodiments, R₁ and R₂ are each alkyl. In embodiments, R₁ and R₂ are each C₁₋₅alkyl. In embodiments, R₁ and R₂ are each independently methyl, ethyl, or isopropyl. In embodiments, R₁ and R₂ are each methyl. In embodiments, R₁ and R₂ taken together with the nitrogen to which they are attached form a 4- to 6-membered heterocyclyl, e.g., azetidine, pyrrolidine, or piperidine.

In embodiments, R₃, R₄, and R₅ are each independently H, D, optionally substituted C₁₋₅alkyl, optionally substituted C₃₋₆cycloalkyl, optionally substituted C₁₋₅heteroalkyl, —P(O)(OC₁₋₅alkyl)₂, —P(O)(OPh)₂, or —P(O)(OH)₂. In embodiments, R₃, R₄, and R₅ are each independently H, optionally substituted C₁₋₅alkyl, optionally substituted C₃₋₆cycloalkyl, optionally substituted C₁₋₅heteroalkyl, —P(O)(OC₁₋₅alkyl)₂, —P(O)(OPh)₂, or —P(O)(OH)₂. In embodiments, R₃, R₄, and R₅ are each independently H, optionally substituted C₁₋₅alkyl, optionally substituted C₃₋₆cycloalkyl, —P(O)(OC₁₋₅alkyl)₂, —P(O)(OPh)₂, or —P(O)(OH)₂. In embodiments, R₃, R₄, and R₅ are each independently H, optionally substituted C₁₋₅alkyl, optionally substituted C₃₋₆cycloalkyl, or —P(O)(OH)₂. In embodiments, R₃, R₄, and R₅ are each independently H, optionally substituted C₁₋₅alkyl, or —P(O)(OH)₂. In embodiments, the C₁₋₅ alkyl is methyl, ethyl, propyl, isopropyl, or t-butyl. In embodiments, the C₁₋₅alkyl is methyl, ethyl, or isopropyl. In embodiments, R₃, R₄, and R₅ are each H.

In embodiments, R₃ is optionally substituted C₁₋₅alkyl or —P(O)(OH)₂, and R₄ and R₅ are each H. In embodiments, R₄ is optionally substituted C₁₋₅alkyl or —P(O)(OH)₂, and R₃ and R₅ are each H. In embodiments, R₅ is optionally substituted C₁₋₅alkyl or —P(O)(OH)₂, and R₃ and R₄ are each H. In embodiments, the C₁₋₅alkyl is methyl, ethyl, propyl, isopropyl, or t-butyl. In embodiments, the C₁₋₅alkyl is methyl, ethyl, or isopropyl.

In embodiments, X is absent, and R₃ is H, halogen, optionally substituted C₁₋₅alkyl, optionally substituted C₃₋₆cycloalkyl, or —P(O)(OH)₂. In embodiments, Y is absent, and R₄ is H, halogen, optionally substituted C₁₋₅alkyl, optionally substituted C₃₋₆cycloalkyl, or —P(O)(OH)₂. In embodiments, Z is absent, and R₃ is H, halogen, optionally substituted C₁₋₅alkyl, optionally substituted C₃₋₆cycloalkyl, or —P(O)(OH)₂. In embodiments, the C₁₋₅alkyl is methyl, ethyl, propyl, isopropyl, or t-butyl. In embodiments, the C₁₋₅alkyl is methyl, ethyl, or isopropyl. In embodiments, the halogen is F, Cl, or Br.

In embodiments, R₆ is H, optionally substituted alkyl, optionally substituted cycloalkyl, optionally substituted alkylene-cycloalkyl, optionally substituted alkylene-heterocyclyl, or optionally substituted alkylene-aryl. In embodiments, R₆ is H, optionally substituted alkyl, optionally substituted cycloalkyl, or optionally substituted alkylene-cycloalkyl. In embodiments, the optionally substituted alkyl is an optionally substituted C₁₋₅alkyl. In embodiments, the optionally substituted cycloalkyl is an optionally substituted C₃₋₆cycloalkyl. In embodiments, the optionally substituted aryl is an optionally substituted phenyl. In embodiments, the optionally substituted heterocyclyl is an optionally substituted 4- to 6-membered heterocyclyl, e.g., an azetidine, pyrrolidine, piperidine, morpholine, thiomorpholine, piperazine, each of which is optionally substituted. In embodiments, the optionally substituted alkylene is an optionally substituted C₁₋₃alkylene. In embodiments, the optionally substituted alkylene is an optionally substituted methylene. In embodiments, R₆ is H, C₁₋₅alkyl, C₃₋₆cycloalkyl or C(═O)(C₁₋₅ alkyl). In embodiments, R₆ is H, C₁₋₅alkyl, or C₃₋₈ cycloalkyl. In embodiments, R₆ is H or C₁₋₅alkyl. In embodiments, R₆ is H. In embodiments, R₆ is C₁₋₅alkyl. In embodiments, the C₁₋₅alkyl is methyl, ethyl, propyl, isopropyl, or t-butyl. In embodiments, the optionally substituted C₁₋₅alkyl is —C(O)—C₁₋₄alkyl, —C(O)—N(H)(C₁₋₄alkyl), or —C(O)—OC₁₋₄alkyl.

In embodiments, R₇ and R₈ are each independently H, halogen, optionally substituted alkyl, —OH, optionally substituted —O-alkyl or optionally substituted —O— cycloalkyl. In embodiments, R₇ and R₈ are each independently H, halogen, optionally substituted C₁₋₅alkyl, —OH, optionally substituted —OC₁₋₅alkyl, or optionally substituted —O—C₃₋₆cycloalkyl. In embodiments, R₇ and R₈ are each independently H or optionally substituted C₁₋₅alkyl. In embodiments, R₇ and R₈ are each H. In embodiments, R₇ and R₈ are each optionally substituted C₁₋₅alkyl. In embodiments, R₇ and R₈ are each halogen. In embodiments, the halogen is F. In embodiments, the C₁₋₅alkyl is methyl, ethyl, or isopropyl. In embodiments, the C₁₋₅alkyl is methyl. In embodiments, the C₁₋₅alkyl is ethyl.

In embodiments, R₇ and R₈ taken together with the atom to which they are attached form an oxo or an optionally substituted cycloalkyl ring. In embodiments, R₇ and R₈ taken together with the atom to which they are attached form an oxo. In embodiments, R₇ and R₈ taken together with the atom to which they are attached form an optionally substituted cycloalkyl ring. In embodiments, the optionally substituted cycloalkyl ring is an optionally substituted C₃₋₆cycloalkyl ring. In embodiments, the optionally substituted cycloalkyl ring is an optionally substituted cyclopropyl. In embodiments, R₇ is H and R₅ is optionally substituted C₁₋₅alkyl. In embodiments, R₇ is H and R₅ is F. In embodiments, R₇ is H and R₅ is —OH. In embodiments, R₇ is H and R₅ is —OCH₃. In embodiments, R₇ is F and R₅ is F. In embodiments, R₇ and R₈ are each optionally substituted C₁₋₅alkyl. In embodiments, the C₁₋₅alkyl is methyl.

In embodiments, R₉ and R₁₀ are each independently H, halogen, optionally substituted alkyl, —OH, optionally substituted —O-alkyl or optionally substituted —O— cycloalkyl. In embodiments, R₉ and R₁₀ are each independently H, halogen, optionally substituted C₁₋₅alkyl, —OH, optionally substituted —OC₁₋₅alkyl, or optionally substituted —O—C₃₋₆cycloalkyl. In embodiments, R₉ and R₁₀ are each independently H or optionally substituted C₁₋₅alkyl. In embodiments, R₉ and R₁₀ are each H. In embodiments, R₉ and R₁₀ are each optionally substituted C₁₋₅alkyl. In embodiments, R₉ and R₁₀ are each halogen. In embodiments, the halogen is F. In embodiments, the C₁₋₅alkyl is methyl, ethyl, or isopropyl. In embodiments, the C₁₋₅alkyl is methyl. In embodiments, the C₁₋₅alkyl is ethyl.

In embodiments, R₉ and R₁₀ taken together with the atom to which they are attached form an optionally substituted cycloalkyl ring. In embodiments, the optionally substituted cycloalkyl ring is an optionally substituted C₃₋₆cycloalkyl ring. In embodiments, the optionally substituted cycloalkyl ring is an optionally substituted cyclopropyl. In embodiments, R₉ is H and R₁₀ is optionally substituted C₁₋₅alkyl. In embodiments, R₉ is H and R₁₀ is F. In embodiments, R₉ is H and R₁₀ is —OH. In embodiments, R₉ is H and R₁₀ is —OCH₃. In embodiments, R₉ and R₁₀ are each F. In embodiments, R₉ and R₁₀ are each optionally substituted C₁₋₅alkyl. In embodiments, the C₁₋₅alkyl is methyl.

In embodiments of Formula (I), R₁₁ and R₁₂ are each independently O⁻, —OH, optionally substituted —O-alkyl, optionally substituted —O-cycloalkyl, optionally substituted —O-alkylene-cycloalkyl, optionally substituted —O-aryl, or —OM, wherein M is a pharmaceutically acceptable cation. In embodiments, the optionally substituted —O-alkyl is an optionally substituted —O-haloalkyl, a hemiaminal, an acetal, or an amino acid residue. In embodiments, R₁₁ and R₁₂ are each independently O⁻, —OH, optionally substituted —O—C₁₋₅alkyl, optionally substituted —O—C₁₋₅haloalkyl, optionally substituted —O—C₃₋₆cycloalkyl, or -OM. In embodiments, R₁₁ and R₁₂ are each independently O⁻, —OH, optionally substituted —O—C₁₋₅alkyl, optionally substituted —O—C₁₋₅haloalkyl, or -OM. In embodiments, R₁₁ is O⁻ and R₁₂ is —OH, optionally substituted —O—C₁₋₅alkyl, optionally substituted —O—C₁₋₅haloalkyl, or -OM. In embodiments, R₁₁ and R₁₂ are each optionally substituted —O—C₁₋₅alkyl. In embodiments, R₁₁ and R₁₂ are each optionally substituted —O—C₁₋₃alkyl. In embodiments, R₁₁ and R₁₂ are each independently —OMe, —OEt, —OiPr, or -OtBu. In embodiments, R₁₁ and R₁₂ are each optionally substituted —O—C₁₋₅haloalkyl. In embodiments, the —O—C₁₋₅haloalkyl is —OCH₃, —OCH₂F, —OCHF₂, or —OCH₂CF₃. In embodiments, the —O—C₁₋₅haloalkyl is a —OCH₂CF₃. In embodiments, M is Na⁺, K⁺, NH₄⁺, or Ca⁺². In embodiments, M is Na⁺, K⁺, or NH₄⁺.

In embodiments of Formula (I), R₁₁ and R₁₂ taken together with the atoms to which they are attached form a heterocyclyl. In embodiments, R₁₁ and R₁₂ taken together with the atoms to which they are attached form a 5- to 12-membered heterocyclyl. In embodiments, the heterocyclyl has the structure:

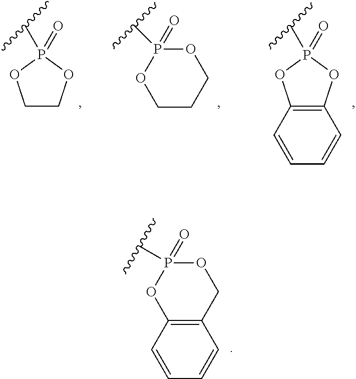 [SMILES: *P1(=O)OCCO1 ; *P1(=O)OCCCO1 ; *P1(=O)Oc2ccccc2O1 ; *P1(=O)OCc2ccccc2O1]

In embodiments of Formula (II), R₁₂ is O⁻, —OH, optionally substituted —O-alkyl, optionally substituted —O-cycloalkyl, optionally substituted —O-alkylene-cycloalkyl, optionally substituted —O-aryl, or —OM, wherein M is a pharmaceutically acceptable cation. In embodiments, the optionally substituted —O-alkyl is an optionally substituted —O-haloalkyl, a hemiaminal, an acetal, or an amino acid residue. In embodiments, R₁₂ is O⁻, —OH, —O-alkyl, —O-haloalkyl, —O-cycloalkyl, or -OM. In embodiments, R₁₂ is O⁻, —OH, optionally substituted —O-alkyl, optionally substituted —O-haloalkyl, or -OM. In embodiments, R₁₂ is O—, optionally substituted —O-alkyl, or optionally substituted —O-haloalkyl. In embodiments, R₁₂ is optionally substituted —O-alkyl. In embodiments, the optionally substituted —O-alkyl is an optionally substituted —O—C₁₋₅alkyl. In embodiments, the —O-alkyl is —OMe, —OEt, —OiPr, or -OtBu. In embodiments, R₁₂ is —O-haloalkyl. In embodiments, the optionally substituted —O-haloalkyl is optionally substituted —O—C₁₋₅haloalkyl. In some embodiments, the —O-haloalkyl is —OCH₂CF₃. In embodiments, R₁₂ is O⁻, —OH, —O—C₁₋₅alkyl, —O—C₁₋₅haloalkyl, —O—C₃₋₆cycloalkyl, or -OM. In embodiments, M is Na⁺, K⁺, NH₄⁺, or Ca⁺². In embodiments, M is Na⁺, K⁺, or NH₄⁺. In embodiments, the —O-alkyl is —OMe, —OEt, —OiPr, or -OtBu. In embodiments, the —O—C₁₋₅haloalkyl is —OCH₃, —OCH₂F, —OCHF₂, or —OCH₂CF₃. In embodiments, the —O—C₁₋₅haloalkyl is —OCH₂CF₃.

In embodiments, R₁₃ is H, D, or C₁₋₅alkyl. In embodiments, R₁₃ is H or —C₁₋₅alkyl. In embodiments, the C₁₋₅alkyl is methyl, ethyl, propyl, isopropyl, or butyl. In embodiments, R₁₃ is H, methyl, ethyl, or isopropyl.

In embodiments of Formula (I), R₁₁ and R₁₃ taken together with the atoms to which they are attached form an optionally substituted heterocyclic ring comprising 4-7 carbon atoms. In embodiments, R₁₁ and R₁₃ taken together with the atoms to which they are attached form an optionally substituted 5-to 7-membered heterocyclic ring. In embodiments, R₁₁ and R₁₃ taken together with the atoms to which they are attached form an optionally substituted 5- or 6-membered heterocyclic ring.

In embodiments, R₁₄ is H, halogen, —OH, optionally substituted alkyl, optionally substituted cycloalkyl, or optionally substituted heteroalkyl. In embodiments, R₁₄ is H, halogen, or optionally substituted alkyl. In embodiments, R₁₄ is H or alkyl. In some embodiments, R₁₄ is H. In embodiments, R₁₄ is alkyl. In embodiments, the alkyl is a C₁-C₅alkyl. In embodiments, the C₁-C₅ alkyl is s methyl, ethyl, or isopropyl. In embodiments, R₁₄ is halogen. In embodiments, the halogen is Cl, Br, or F. In embodiments, the cycloalkyl is a C₃-C₆ cycloalkyl. In embodiments, the cycloalkyl is cyclopropyl. In embodiments, the optionally substituted heteroalkyl is —CH₂—O—C₁₋₅alkyl. In embodiments, the optionally substituted heteroalkyl is —CH₂—N—(H)(C₁₋₅alkyl) or —CH₂—N—(C₁₋₅alkyl)₂.

In embodiments of Formula (I), R₁₅ is H, optionally substituted C₁₋₅alkyl, or optionally substituted phenyl.

In embodiment of Formula (II), n is 0 or 1. In embodiments, n is 0. In some embodiments, n is 1. In embodiments, n is 2.

In embodiments, provided herein is one or more compounds of Formula (I-A) or (I-B) selected from Table 1 or a pharmaceutically acceptable salt thereof.

TABLE 1
Compounds

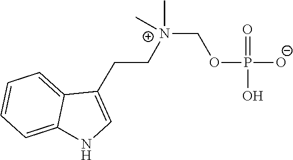 [SMILES: C[N+](C)(CCc1c[nH]c2ccccc12)COP(=O)([O-])O]

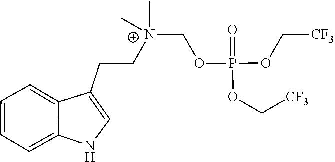 [SMILES: C[N+](C)(CCc1c[nH]c2ccccc12)COP(=O)(OCC(F)(F)F)OCC(F)(F)F]

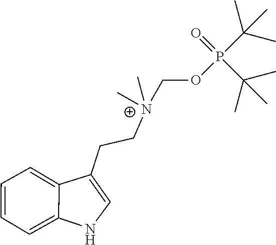 [SMILES: CC(C)(C)P(=O)(OC[N+](C)(C)CCc1c[nH]c2ccccc12)C(C)(C)C]

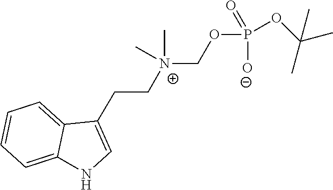 [SMILES: CC(C)(C)OP(=O)([O-])OC[N+](C)(C)CCc1c[nH]c2ccccc12]

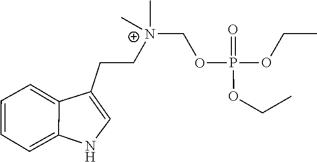 [SMILES: CCOP(=O)(OCC)OC[N+](C)(C)CCc1c[nH]c2ccccc12]

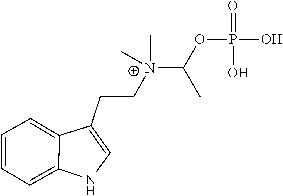 [SMILES: CC(OP(=O)(O)O)[N+](C)(C)CCc1c[nH]c2ccccc12]

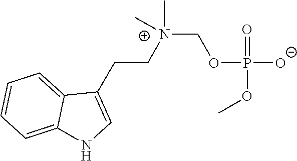 [SMILES: COP(=O)([O-])OC[N+](C)(C)CCc1c[nH]c2ccccc12]

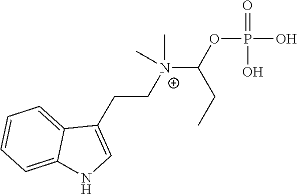 [SMILES: CCC(OP(=O)(O)O)[N+](C)(C)CCc1c[nH]c2ccccc12]

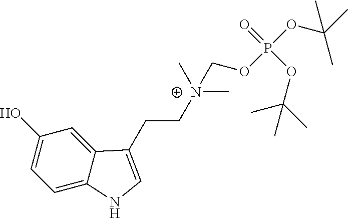 [SMILES: CC(C)(C)OP(=O)(OC[N+](C)(C)CCc1c[nH]c2ccc(O)cc12)OC(C)(C)C]

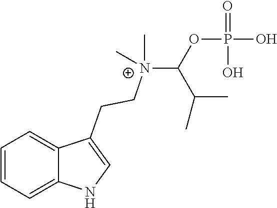 [SMILES: CC(C)C(OP(=O)(O)O)[N+](C)(C)CCc1c[nH]c2ccccc12]

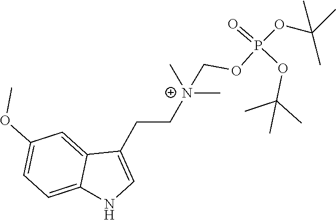 [SMILES: COc1ccc2[nH]cc(CC[N+](C)(C)COP(=O)(OC(C)(C)C)OC(C)(C)C)c2c1]

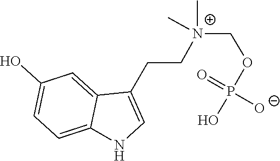 [SMILES: C[N+](C)(CCc1c[nH]c2ccc(O)cc12)COP(=O)([O-])O]

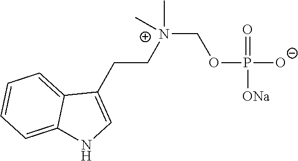

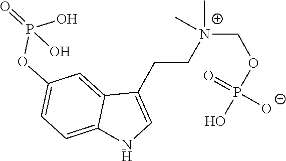 [SMILES: C[N+](C)(CCc1c[nH]c2ccc(OP(=O)(O)O)cc12)COP(=O)([O-])O]

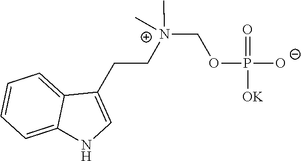

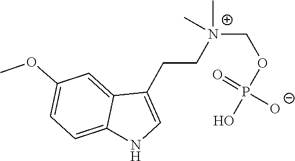 [SMILES: COc1ccc2[nH]cc(CC[N+](C)(C)COP(=O)([O-])O)c2c1]

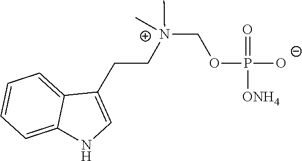

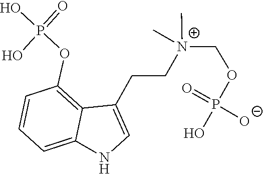 [SMILES: C[N+](C)(CCc1c[nH]c2cccc(OP(=O)(O)O)c12)COP(=O)([O-])O]

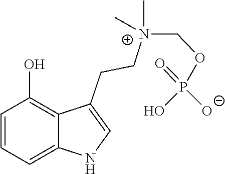 [SMILES: C[N+](C)(CCc1c[nH]c2cccc(O)c12)COP(=O)([O-])O]

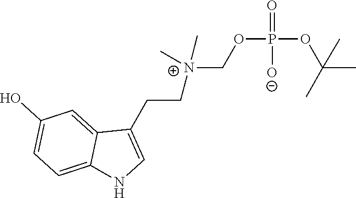 [SMILES: CC(C)(C)OP(=O)([O-])OC[N+](C)(C)CCc1c[nH]c2ccc(O)cc12]

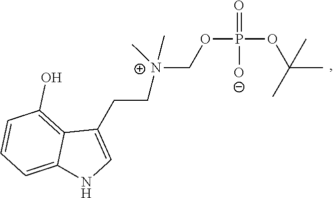 [SMILES: CC(C)(C)OP(=O)([O-])OC[N+](C)(C)CCc1c[nH]c2cccc(O)c12]

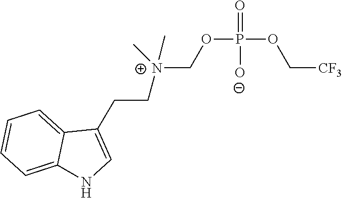 [SMILES: C[N+](C)(CCc1c[nH]c2ccccc12)COP(=O)([O-])OCC(F)(F)F]

or

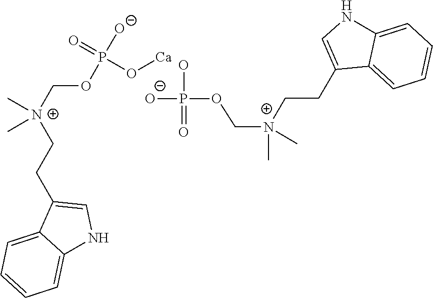

In embodiments, provided herein is one or more compounds of Formula (I-A) or (I-B) selected from Table 2 or a pharmaceutically acceptable salt thereof.

TABLE 2
Compounds

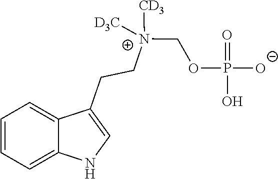 [SMILES: [2H]C([2H])([2H])[N+](CCc1c[nH]c2ccccc12)(COP(=O)([O-])O)C([2H])([2H])[2H]]

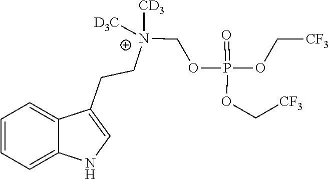 [SMILES: [2H]C([2H])([2H])[N+](CCc1c[nH]c2ccccc12)(COP(=O)(OCC(F)(F)F)OCC(F)(F)F)C([2H])([2H])[2H]]

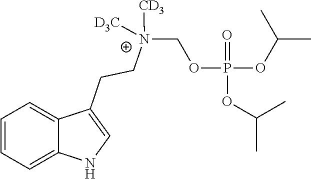 [SMILES: [2H]C([2H])([2H])[N+](CCc1c[nH]c2ccccc12)(COP(=O)(OC(C)C)OC(C)C)C([2H])([2H])[2H]]

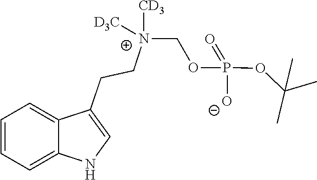 [SMILES: [2H]C([2H])([2H])[N+](CCc1c[nH]c2ccccc12)(COP(=O)([O-])OC(C)(C)C)C([2H])([2H])[2H]]

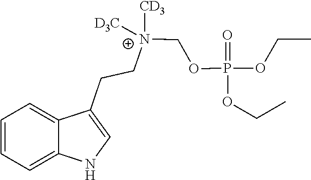 [SMILES: [2H]C([2H])([2H])[N+](CCc1c[nH]c2ccccc12)(COP(=O)(OCC)OCC)C([2H])([2H])[2H]]

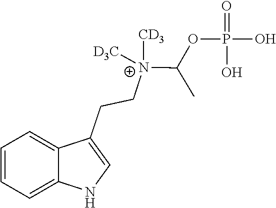 [SMILES: [2H]C([2H])([2H])[N+](CCc1c[nH]c2ccccc12)(C(C)OP(=O)(O)O)C([2H])([2H])[2H]]

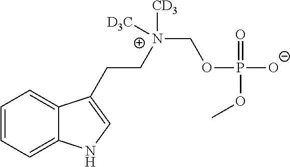 [SMILES: [2H]C([2H])([2H])[N+](CCc1c[nH]c2ccccc12)(COP(=O)([O-])OC)C([2H])([2H])[2H]]

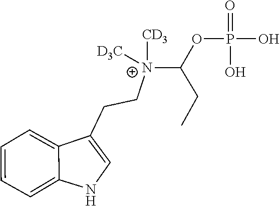 [SMILES: [2H]C([2H])([2H])[N+](CCc1c[nH]c2ccccc12)(C(CC)OP(=O)(O)O)C([2H])([2H])[2H]]

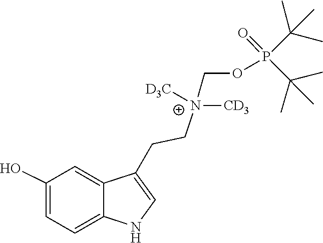 [SMILES: [2H]C([2H])([2H])[N+](CCc1c[nH]c2ccc(O)cc12)(COP(=O)(C(C)(C)C)C(C)(C)C)C([2H])([2H])[2H]]

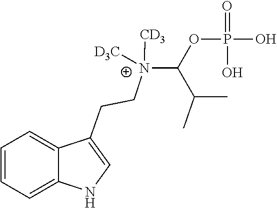

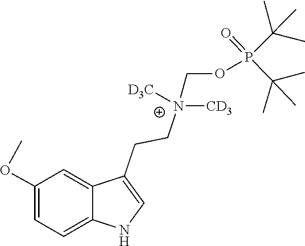 [SMILES: [2H]C([2H])([2H])[N+](CCc1c[nH]c2ccc(OC)cc12)(COP(=O)(C(C)(C)C)C(C)(C)C)C([2H])([2H])[2H]]

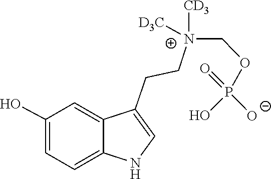 [SMILES: [2H]C([2H])([2H])[N+](CCc1c[nH]c2ccc(O)cc12)(COP(=O)([O-])O)C([2H])([2H])[2H]]

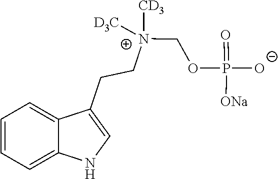

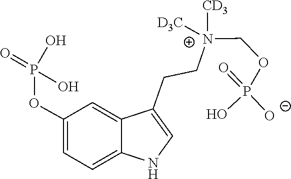

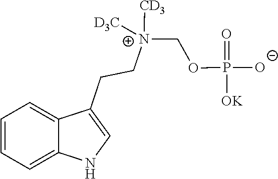

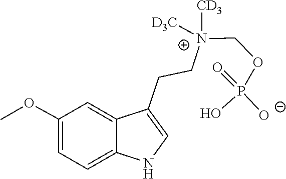 [SMILES: [2H]C([2H])([2H])[N+](CCc1c[nH]c2ccc(OC)cc12)(COP(=O)([O-])O)C([2H])([2H])[2H]]

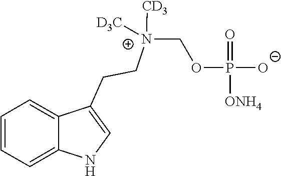

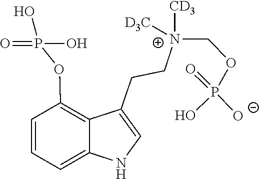 [SMILES: [2H]C([2H])([2H])[N+](CCc1c[nH]c2cccc(OP(=O)(O)O)c12)(COP(=O)([O-])O)C([2H])([2H])[2H]]

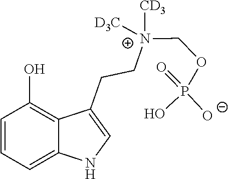 [SMILES: [2H]C([2H])([2H])[N+](CCc1c[nH]c2cccc(O)c12)(COP(=O)([O-])O)C([2H])([2H])[2H]]

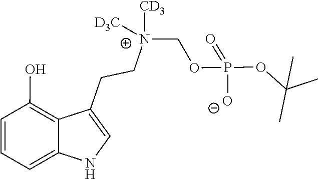

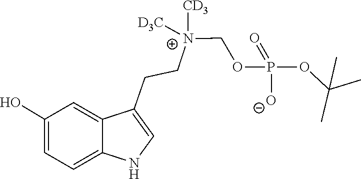 [SMILES: [2H]C([2H])([2H])[N+](CCc1c[nH]c2ccc(O)cc12)(COP(=O)([O-])OC(C)(C)C)C([2H])([2H])[2H]]

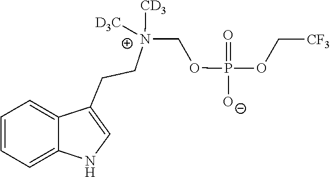 [SMILES: [2H]C([2H])([2H])[N+](CCc1c[nH]c2ccccc12)(COP(=O)([O-])OCC(F)(F)F)C([2H])([2H])[2H]]

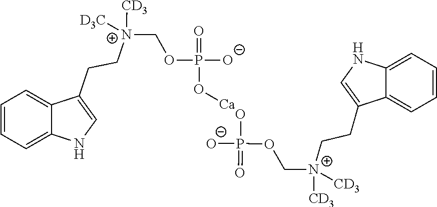

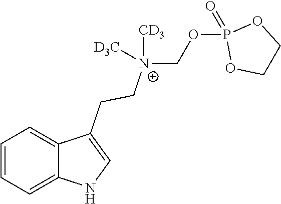 [SMILES: [2H]C([2H])([2H])[N+](CCc1c[nH]c2ccccc12)(COP1(=O)OCCO1)C([2H])([2H])[2H]]

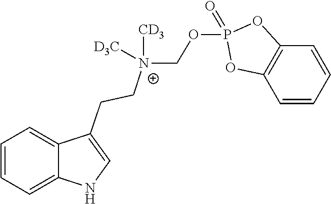 [SMILES: [2H]C([2H])([2H])[N+](CCc1c[nH]c2ccccc12)(COP1(=O)Oc2ccccc2O1)C([2H])([2H])[2H]]

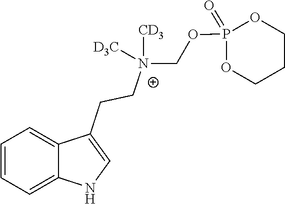 [SMILES: [2H]C([2H])([2H])[N+](CCc1c[nH]c2ccccc12)(COP1(=O)OCCCO1)C([2H])([2H])[2H]]

In embodiments, provided herein is one or more compounds of Formula (II-A) or (II-B) selected from Table 3 or a pharmaceutically acceptable salt thereof.

TABLE 3
Compounds

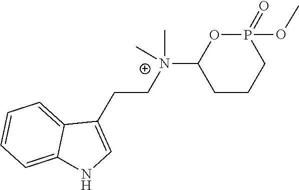 [SMILES: COP1(=O)CCCC([N+](C)(C)CCc2c[nH]c3ccccc23)O1]

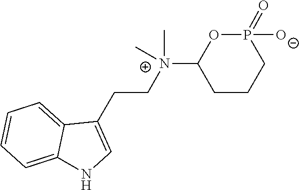 [SMILES: C[N+](C)(CCc1c[nH]c2ccccc12)C1CCCP(=O)([O-])O1]

In embodiments, provided herein is one or more compounds of Formula (II-A) or (II-B) selected from Table 4 or a pharmaceutically acceptable salt thereof.

TABLE 4
Compounds

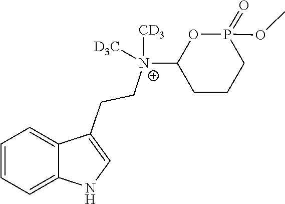 [SMILES: [2H]C([2H])([2H])[N+](CCc1c[nH]c2ccccc12)(C1CCCP(=O)(OC)O1)C([2H])([2H])[2H]]

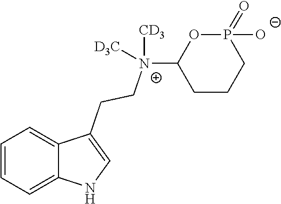 [SMILES: [2H]C([2H])([2H])[N+](CCc1c[nH]c2ccccc12)(C1CCCP(=O)([O-])O1)C([2H])([2H])[2H]]

In embodiments, provided herein is a compound of Formula (I-A), (I-B), (II-A), or (II-B) as described herein, wherein the Formulas (I-A), (I-B), (II-A), and (II-B) exclude the compounds described in International Application No. PCT/US2022/032918.

### Compositions

In embodiments of the present disclosure, a pharmaceutical composition comprises a therapeutically effective amount of one or more compounds of the present disclosure (e.g., compounds of Formula (I-A), Formula (I-B), Formula (II-A), Formula (II-B) or Compounds of Table 1, Table 2, Table 3, or Table 4) or a pharmaceutically acceptable salt thereof and a pharmaceutically acceptable excipient.

The pharmaceutically acceptable excipients and adjuvants are added to the composition or formulation for a variety of purposes. In embodiments, a pharmaceutical composition comprising one or more compounds disclosed herein, or a pharmaceutically acceptable salt thereof, further comprise a pharmaceutically acceptable carrier. In embodiments, a pharmaceutically acceptable carrier includes a pharmaceutically acceptable excipient, binder, and/or diluent. In embodiments, suitable pharmaceutically acceptable carriers include, but are not limited to, inert solid fillers or diluents and sterile aqueous or organic solutions. In embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

### Methods of Use

In one aspect, the present disclosure provides methods of treating or preventing neurological disorders in a subject in need thereof, the methods comprising administering a therapeutically effective amount of a compound disclosed herein (e.g., compounds of Formula (I-A), Formula (I-B), Formula (II-A), Formula (II-B) or Compounds of Table 1, Table 2, Table 3, or Table 4), a pharmaceutically acceptable salt thereof, or a pharmaceutical composition thereof to the subject.

In embodiments, the neurological disorder is a mood disorder. In embodiments, the mood disorder is clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, major depressive disorder, minor depressive disorder, bipolar disorder or manic depressive disorder, depression caused by chronic medical conditions, treatment-resistant depression, refractory depression, suicidality, suicidal ideation, or suicidal behavior. In embodiments, the method described herein provides therapeutic effect to a subject suffering from depression (e.g., moderate or severe depression). In embodiments, the mood disorder is associated with neuroendocrine diseases and disorders, neurodegenerative diseases and disorders (e.g., epilepsy), movement disorders, tremor (e.g., Parkinson's Disease), or women's health disorders or conditions. In embodiments, the mood disorder is depression. In embodiments, the mood disorder is treatment-resistant depression or major depressive disorder. In embodiments, the mood disorder is major depressive disorder. In embodiments, the mood disorder is treatment-resistant depression.

In embodiments, the present disclosure provides methods of treating or preventing PTSD, mood disorders, general anxiety disorder, addictive disorders, and/or drug dependence in a subject in need thereof, the methods comprising administering a therapeutically effective amount of a compound disclosed herein (e.g., compounds of Formula (I-A), Formula (I-B), Formula (II-A), Formula (II-B) or Compounds of Table 1, Table 2, Table 3, or Table 4), a pharmaceutically acceptable salt thereof, or a pharmaceutical composition thereof to the subject.

In embodiments, the present disclosure provides methods of treating or preventing PTSD in a subject in need thereof, the methods comprising administering a therapeutically effective amount of a compound disclosed herein (e.g., compounds of Formula (I-A), Formula (I-B), Formula (II-A), Formula (II-B) or Compounds of Table 1, Table 2, Table 3, or Table 4), a pharmaceutically acceptable salt thereof, or a pharmaceutical composition thereof to the subject.

In embodiments, the methods include treating PTSD through induction and maintenance therapy by administering a compound disclosed herein, a pharmaceutically acceptable salt thereof, or a pharmaceutical composition thereof. In embodiments, the compounds of the present disclosure are used for induction and maintenance therapy to treat PTSD with an improved safety profile when compared to treatment with the entactogenic, oneirophrenic or psychedelic compound (e.g., dimethyltryptamine or related compound, psilocybin, or MDMA) alone.

In embodiments, the present disclosure provides methods of treating or preventing behavioral or mood disorders in a subject in need thereof, the methods comprising administering a therapeutically effective amount of a compound disclosed herein (e.g., compounds of Formula (I-A), Formula (I-B), Formula (II-A), Formula (II-B) or Compounds of Table 1, Table 2, Table 3, or Table 4), a pharmaceutically acceptable salt thereof, or a pharmaceutical composition thereof to the subject. In embodiments, the behavioral or mood disorder includes anxiety, such as social anxiety in autistic subjects (e.g., autistic adults) and anxiety related to life-threatening illnesses. In embodiments, the behavioral or mood disorder includes stress (where moderation thereof is measured, e.g., by effects on amygdala responses). In embodiments, the anxiety disorder is panic disorder, obsessive-compulsive disorder, and/or general anxiety disorder. In embodiments, the subject suffers from a lack of motivation, attention, lack of accuracy in memory recall, speed of response, perseveration, and/or cognitive engagement. Further examples include depression (e.g., MDD or TRD), attention disorders, disorders of executive function and/or cognitive engagement, obsessive compulsive disorder, bipolar disorder, panic disorder, phobia, schizophrenia, psychopathy, antisocial personality disorder and/or neurocognitive disorders.

In embodiments, the present disclosure provides methods for treating an addictive disorder in a subject in need thereof, the methods comprising administering a therapeutically effective amount of a compound disclosed herein (e.g., compounds of Formula (I-A), Formula (I-B), Formula (II-A), Formula (II-B) or Compounds of Table 1, Table 2, Table 3, or Table 4), a pharmaceutically acceptable salt thereof, or a pharmaceutical composition thereof to the subject. In embodiments, the addictive disorder is alcohol abuse, substance abuse, smoking, obesity, or mixtures thereof. In embodiments, the disorder is an eating disorder (e.g., anorexia nervosa, bulimia, nervosa, binge eating disorder, etc.) or an auditory disorder.

In embodiments, the present disclosure provides methods for treating an impulsive disorder in a subject in need thereof, the methods comprising administering a therapeutically effective amount of a compound disclosed herein (e.g., compounds of Formula (I-A), Formula (I-B), Formula (II-A), Formula (II-B) or Compounds of Table 1, Table 2, Table 3, or Table 4), a pharmaceutically acceptable salt thereof, or a pharmaceutical composition thereof to the subject. In embodiments, the impulsive disorder is attention deficit hyperactivity disorder (ADHD), attention deficit disorder (ADD), Tourette's syndrome, autism, or combinations thereof.

In embodiments, the present disclosure provides methods for treating a compulsive disorder in a subject in need thereof, the methods comprising administering a therapeutically effective amount of a compound disclosed herein (e.g., compounds of Formula (I-A), Formula (I-B), Formula (II-A), Formula (II-B) or Compounds of Table 1, Table 2, Table 3, or Table 4), a pharmaceutically acceptable salt thereof, or a pharmaceutical composition thereof to the subject. In embodiments, the compulsive disorder is obsessive compulsive disorder (OCD), gambling, aberrant sexual behavior, or combinations thereof.

In embodiments, the present disclosure provides methods for treating a personality disorder in a subject in need thereof, the methods comprising administering a therapeutically effective amount of a compound disclosed herein (e.g., compounds of Formula (I-A), Formula (I-B), Formula (II-A), Formula (II-B) or Compounds of Table 1, Table 2, Table 3, or Table 4), a pharmaceutically acceptable salt thereof, or a pharmaceutical composition thereof. In embodiments, the personality disorder is conduct disorder, antisocial personality, aggressive behavior, or combinations thereof to the subject.

### NUMBERED EMBODIMENTS

1. A compound of Formula (I-B)

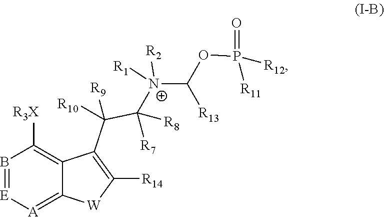

or a pharmaceutically acceptable salt thereof,
wherein:
A is N or CH;
B is N or C—YR₄;
E is N or C—ZR₅, wherein no more than two of A, B, and E are N;
W is O, S, or NR₆;
X, Y, and Z are each independently absent, O, S, or NH;
R₁ and R₂ are each independently H, D, optionally substituted alkyl, optionally substituted cycloalkyl, or optionally substituted heteroalkyl;
R₃, R₄, and R₅ are each independently H, D, optionally substituted alkyl, optionally substituted cycloalkyl, optionally substituted heteroalkyl, or —P(O)(OH)₂;
R₆ is H, optionally substituted alkyl, optionally substituted cycloalkyl, alkylene-optionally substituted cycloalkyl, or optionally substituted heteroalkyl;
R₇ and R₈ are each independently H, D, F, optionally substituted alkyl, optionally substituted cycloalkyl, or optionally substituted heteroalkyl, or R₇ and R₈ taken together with the atom to which they are attached form an optionally substituted cycloalkyl ring;
R₉ and R₁₀ are each independently H, D, halogen, optionally substituted alkyl, optionally substituted cycloalkyl, or optionally substituted heteroalkyl, or R₉ and R₁₀ taken together with the atom to which they are attached form an oxo;
R₁₁ and R₁₂ are each independently O⁻, —OH, —OD, —O-alkyl, —O-cycloalkyl, —O-alkylene-cycloalkyl, —O-aryl, or —OM, wherein M is a pharmaceutically acceptable cation, or R₁₁ and R₁₂ together with the atoms to which they are attached form a heterocyclyl, and the alkyl, cycloalkyl, aryl and heterocyclyl are each optionally substituted;
R₁₃ is H, D, halogen, optionally substituted alkyl, optionally substituted alkoxy, optionally substituted heteroalkyl, optionally substituted aryl, optionally substituted heteroaryl, alkylene-optionally substituted aryl, —C(O)-(alkylene-optionally substituted aryl), or —O—C(O)—NHR₁₅, or R₁₁ and R₁₃ taken together with the atoms to which they are attached form an optionally substituted heterocyclic ring comprising 4-7 carbon atoms;
R₁₄ is H, D, halogen, —OH, an optionally substituted alkyl, optionally substituted cycloalkyl, or optionally substituted heteroalkyl; and
R₁₅ is H, optionally substituted alkyl, optionally substituted cycloalkyl, optionally substituted aryl, or optionally substituted heteroaryl.
   2. The compound of embodiment 1, wherein A is CH.
   3. The compound of embodiment 1 or 2, wherein B in C—YR₄.
   4. The compound of any one of embodiments 1-3, wherein Y is absent.
   5. The compound of any one of embodiments 1-3, wherein Y is O.
   6. The compound of any one of embodiments 1-5, wherein R₄ is H, C₁₋₅alkyl, C₃₋₆cycloalkyl, alkylene- C₃₋₆cycloalkyl, or —P(O)(OH)₂.
   7. The compound of any one of embodiments 1-6, wherein R₄ is H, C₁₋₅alkyl, or —P(O)(OH)₂.
   8. The compound of any one of embodiments 1-7, wherein E is C—ZR₅.
   9. The compound of any one of embodiments 1-8, wherein Z is absent.
   10. The compound of any one of embodiments 1-8, wherein Z is O.
   11. The compound of any one of embodiments 1-10, wherein R₅ is H, C₁₋₅alkyl, C₃₋₆cycloalkyl, alkylene-C₃₋₆cycloalkyl, or —P(O)(OH)₂.
   12. The compound of any one of embodiments 1-10, wherein R₅ is H or C₁₋₅alkyl.
   13. The compound of any one of embodiments 1-10, wherein R₅ is H.
   14. The compound of any one of embodiments 1-7, wherein E is N.
   15. The compound of any one of embodiments 1, 2, and 8-13, wherein B is N.
   16. The compound of any one of embodiments 1-15, wherein X is absent.
   17. The compound of any one of embodiments 1-15, wherein X is O.
   18. The compound of any one of embodiments 1-17, wherein R₃ is H, alkyl, cycloalkyl, alkylene-cycloalkyl, or —P(O)(OH)₂.
   19. The compound of any one of embodiments 1-18, wherein R₃ is H, C₁₋₅alkyl, or —P(O)(OH)₂.
   20. The compound of any one of embodiments 1-19, wherein R₃ is H.
   21. The compound of any one of embodiments 1-20, wherein W is NR₆.
   22. The compound of any one of embodiments 1-21, wherein R₆ is H, C₁₋₅alkyl, C₃₋₆cycloalkyl, or alkylene-C₃₋₆cycloalkyl.
   23. The compound of any one of embodiments 1-22, wherein R₆ is H or C₁₋₅alkyl.
   24. The compound of any one of embodiments 1-23, wherein R₆ is H.
   25. The compound of any one of embodiments 1-24, wherein R₁ and R₂ are each independently H or alkyl.
   26. The compound of any one of embodiments 1-25, wherein R₁ and R₂ are each alkyl.
   27. The compound of embodiment 25 or 26, wherein the alkyl is a C₁₋₅alkyl.
   28. The compound of embodiment 25 or 26, wherein the alkyl is methyl, ethyl, or isopropyl.
   29. The compound of any one of embodiments 1-28, wherein R₇ and R₈ are each independently H, F, or alkyl.
   30. The compound of any one of embodiments 1-29, wherein R₇ and R₈ are each independently H or alkyl.
   31. The compound of any one of embodiments 1-29, wherein R₇ and R₈ are each alkyl.
   32. The compound of any one of embodiments 29-31, wherein the alkyl is a C₁₋₅alkyl.
   33. The compound of any one of embodiments 29-31, wherein the alkyl is a methyl.
   34. The compound of any one of embodiments 1-29, wherein R₇ and R₈ are each independently H or F.
   35. The compound of any one of embodiments 1-29, wherein R₇ and R₈ are each H.
   36. The compound of any one of embodiments 1-29, wherein R₇ is H and R₅ is F.
   37. The compound of any one of embodiments 1-28, wherein R₇ and R₈ taken together with the atom to which they are attached form a C₃₋₆cycloalkyl.
   38. The compound of any one of embodiments 1-28, wherein R₇ and R₈ taken together with the atom to which they are attached form a cyclopropyl.
   39. The compound of any one of embodiments 1-38, wherein R₉ and R₁₀ are each independently H, F, or C₁₋₅alkyl.
   40. The compound of any one of embodiments 1-39, wherein R₉ and R₁₀ are each independently H or C₁₋₅alkyl.
   41. The compound of any one of embodiments 1-39, wherein R₉ and R₁₀ are each C₁₋₅alkyl.
   42. The compound of any one of embodiments 1-39, wherein the R₉ and R₁₀ are each C₁₋₃alkyl.
   43. The compound of any one of embodiments 1-39, wherein the R₉ and R₁₀ are each independently H or methyl.
   44. The compound of any one of embodiments 1-39, wherein R₉ and R₁₀ are each independently H or F.
   45. The compound of any one of embodiments 1-39, wherein R₉ and R₁₀ are each H.
   46. The compound of any one of embodiments 1-39, wherein R₉ is H and R₁₀ is F.
   47. The compound of any one of embodiments 1-38, wherein R₉ and R₁₀ taken together with the atom to which they are attached form an oxo.
   48. The compound of any one of embodiments 1-47, wherein R₁₁ and R₁₂ are each independently O⁻, —OH, —O—C₁₋₅alkyl, —O—C₁₋₅haloalkyl, —O— C₃₋₆cycloalkyl, or -OM.
   49. The compound of any one of embodiments 1-48, wherein R₁₁ and R₁₂ are each independently O⁻, —OH, —O—C₁₋₅alkyl, —O—C₁₋₅haloalkyl, or -OM.
   50. The compound of any one of embodiments 1-49, wherein R₁₁ is O⁻ and R₁₂ is —OH, —O—C₁₋₅alkyl, —O—C₁₋₅haloalkyl, or -OM.
   51. The compound of any one of embodiments 1-49, wherein R₁₁ and R₁₂ are each —O—C₁₋₅alkyl.
   52. The compound of any one of embodiments 1-49, wherein R₁₁ and R₁₂ are each —O—C₁₋₃alkyl.
   53. The compound of any one of embodiments 1-49, wherein R₁₁ and R₁₂ are each independently —OMe, —OEt, —OiPr, or -OtBu.
   54. The compound of any one of embodiments 1-49, wherein R₁₁ and R₁₂ are each —O—C₁₋₅haloalkyl.
   55. The compound of any one of embodiments 48-50 and 54, wherein the —O—C₁₋₅haloalkyl is a —OCH₂CF₃.
   56. The compound of any one of embodiments 1-50, wherein M is Na⁺, K⁺, or NH₄⁺.
   57. The compound of any one of embodiments 1-47, wherein R₁₁ and R₁₂ taken together with the atoms to which they are attached form a heterocyclyl.
   58. The compound of any one of embodiments 1-47, wherein R₁₁ and R₁₂ taken together with the atoms to which they are attached form a 5- to 12-membered heterocyclyl.
   59. The compound of embodiment 57 or 58, wherein the heterocyclyl is:

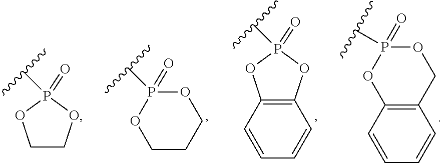

60. The compound of any one of embodiments 1-59, wherein R₁₃ is H, D, or C₁₋₅alkyl.
61. The compound of any one of embodiments 1-60, wherein R₁₃ is H or C₁₋₅alkyl.
62. The compound of any one of embodiments 1-60, wherein R₁₃ is H, methyl, ethyl, or isopropyl.
63. The compound of any one of embodiments 1-60, wherein R₁₄ is H, F, Cl, Br or methyl.
64. The compound of any one of embodiments 1-63, wherein R₁₄ is H.
65. The compound of embodiment 1, having the structure:

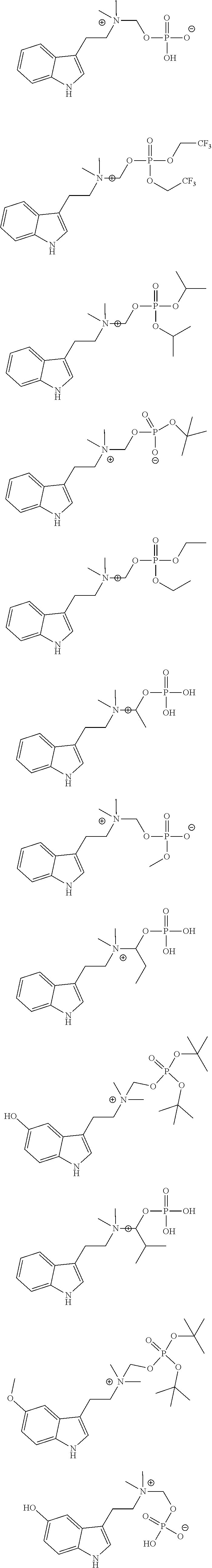

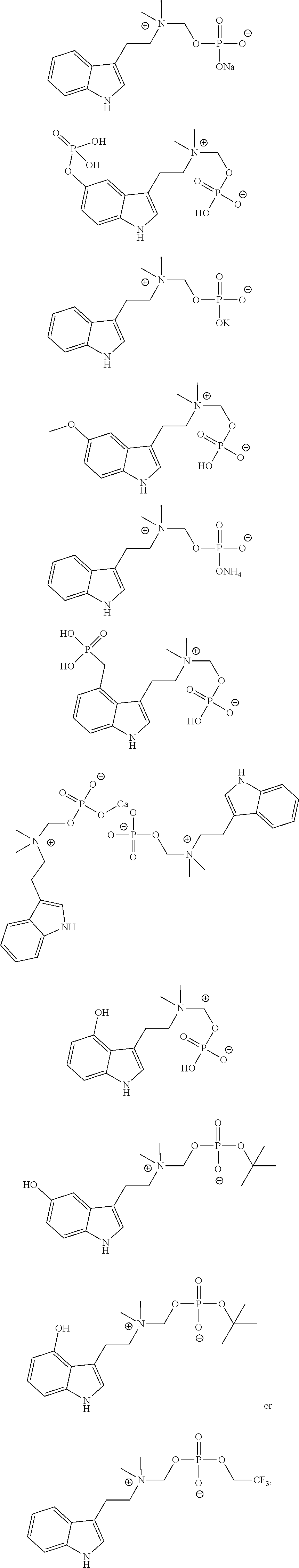

or a pharmaceutically acceptable salt thereof.

66. The compound of embodiment 1, having the structure:

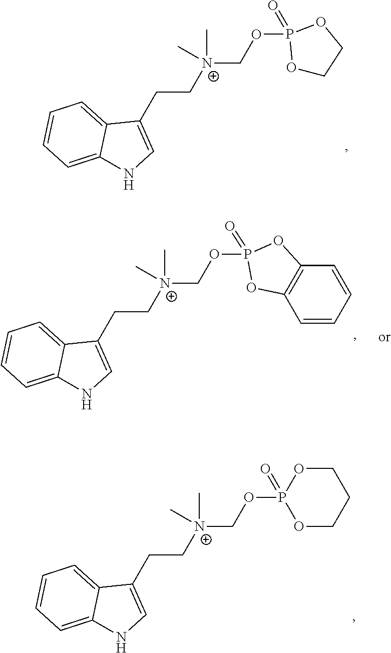 [SMILES: C[N+](C)(CCc1c[nH]c2ccccc12)COP1(=O)OCCO1 ; C[N+](C)(CCc1c[nH]c2ccccc12)COP1(=O)Oc2ccccc2O1 ; C[N+](C)(CCc1c[nH]c2ccccc12)COP1(=O)OCCCO1]

or a pharmaceutically acceptable salt thereof,

67. A compound of Formula (II-B):

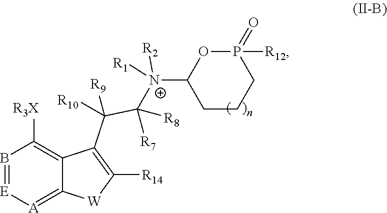

or a pharmaceutically acceptable salt thereof,
wherein:
A is N or CH;
B is N or C—YR₄;
E is N or C—ZR₅, wherein no more than two of A, B, and E are N;
W is O, S, or NR₆;
X, Y, and Z are each independently absent, O, S, or NH;
R₁ and R₂ are each independently D, optionally substituted alkyl, optionally substituted cycloalkyl, or optionally substituted heteroalkyl;
R₃, R₄, and R₅ are each independently H, D, optionally substituted alkyl, optionally substituted cycloalkyl, optionally substituted heteroalkyl, or —P(O)(OH)₂;
R₆ is H, optionally substituted alkyl, optionally substituted cycloalkyl, optionally substituted alkylene-cycloalkyl, or optionally substituted heteroalkyl;
R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;
R₉ and R₁₀ are each independently H, D, halogen, —OH, optionally substituted alkyl, optionally substituted cycloalkyl, or optionally substituted heteroalkyl, or R₉ and R₁₀ taken together with the atom to which they are attached form an oxo;
R₁₂ is O⁻, —OH, —OD, —O-alkyl, —O-cycloalkyl, —O-alkylene-cycloalkyl, —O-aryl, or —OM, wherein M is a pharmaceutically acceptable cation, and the alkyl, cycloalkyl, aryl and heterocyclyl are each optionally substituted;
R₁₄ is H, D, halogen, —OH, optionally substituted alkyl, optionally substituted cycloalkyl, or optionally substituted heteroalkyl; and
n is 0, 1, or 2.
   68. The compound of embodiment 67, wherein A is CH.
   69. The compound of embodiment 67 or 68, wherein B in C-YR₄.
   70. The compound of any one of embodiments 67-69, wherein Y is absent.
   71. The compound of any one of embodiments 67-69, wherein Y is O.
   72. The compound of any one of embodiments 67-71, wherein R₄ is H, C₁₋₅alkyl, C₃₋₆cycloalkyl, alkylene-C₃₋₆cycloalkyl, or —P(O)(OH)₂.
   73. The compound of any one of embodiments 67-72, wherein R₄ is H, C₁₋₅alkyl, or —P(O)(OH)₂.
   74. The compound of any one of embodiments 67-73, wherein E is C—ZR₅.
   75. The compound of any one of embodiments 67-74, wherein Z is absent.
   76. The compound of any one of embodiments 67-74, wherein Z is O.
   77. The compound of any one of embodiments 67-76, wherein R₅ is H, C₁₋₅alkyl, C₃₋₆cycloalkyl, alkylene-C₃₋₆cycloalkyl, or —P(O)(OH)₂.
   78. The compound of any one of embodiments 67-76, wherein R₅ is H or C₁₋₅alkyl.
   79. The compound of any one of embodiments 67-76, wherein R₅ is H.
   80. The compound of any one of embodiments 67-73, wherein E is N.
   81. The compound of any one of embodiments 67, 68, and 74-79, wherein B is N.
   82. The compound of any one of embodiments 67-81, wherein X is absent.
   83. The compound of any one of embodiments 67-81, wherein X is O.
   84. The compound of any one of embodiments 67-83, wherein R₃ is H, C₁₋₅alkyl, C₃₋₆cycloalkyl, alkylene-C₃₋₆cycloalkyl, or —P(O)(OH)₂.
   85. The compound of any one of embodiments 67-84, wherein R₃ is H, C₁₋₅alkyl, or —P(O)(OH)₂.
   86. The compound of any one of embodiments 67-85, wherein R₃ is H.
   87. The compound of any one of embodiments 67-86, wherein W is NR₆.
   88. The compound of any one of embodiments 67-87, wherein R₆ is H, C₁₋₅alkyl, C₃₋₆cycloalkyl, or alkylene-C₃₋₆cycloalkyl.
   89. The compound of any one of embodiments 67-88, wherein R₆ is H or C₁₋₅alkyl.
   90. The compound of any one of embodiments 67-89, wherein R₆ is H.
   91. The compound of any one of embodiments 67-90, wherein R₁ and R₂ are each independently H or alkyl.
   92. The compound of any one of embodiments 67-91, wherein R₁ and R₂ are each alkyl.
   93. The compound of embodiment 91 or 92, wherein the alkyl is a C₁₋₅alkyl.
   94. The compound of embodiment 91 or 92, wherein the alkyl is methyl, ethyl, or isopropyl.
   95. The compound of any one of embodiments 67-94, wherein R₇ and R₈ are each independently H, F, or alkyl.
   96. The compound of any one of embodiments 67-95, wherein R₇ and R₈ are each independently H or alkyl.
   97. The compound of any one of embodiments 67-95, wherein R₇ and R₈ are each alkyl.
   98. The compound of embodiment 97, wherein the alkyl is a C₁₋₅alkyl.
   99. The compound of embodiment 97 or 98, wherein the alkyl is a methyl.
   100. The compound of any one of embodiments 67-95, wherein R₇ and R₈ are each independently H or F.
   101. The compound of any one of embodiments 67-95, wherein R₇ and R₈ are each H.
   102. The compound of any one of embodiments 67-95, wherein R₇ is H and R₅ is F.
   103. The compound of any one of embodiments 67-94, wherein R₇ and R₈ taken together with the atom to which they are attached form a C₃₋₆cycloalkyl.
   104. The compound of any one of embodiments 67-94, wherein R₇ and R₈ taken together with the atom to which they are attached form a cyclopropyl.
   105. The compound of any one of embodiments 67-104, wherein R₉ and R₁₀ are each independently H, F, or alkyl.
   106. The compound of any one of embodiments 67-105, wherein R₉ and R₁₀ are each independently H or alkyl.
   107. The compound of any one of embodiments 67-105, wherein R₉ and R₁₀ are each alkyl.
   108. The compound of any one of embodiments 105-107, wherein the alkyl is a C₁₋₅alkyl.
   109. The compound of any one of embodiments 105-107, wherein the alkyl is a methyl.
   110. The compound of any one of embodiments 67-105, wherein R₉ and R₁₀ are each independently H or F.
   111. The compound of any one of embodiments 67-105, wherein R₉ and R₁₀ are each H.
   112. The compound of any one of embodiments 67-105, wherein R₉ is H and R₁₀ is F.
   113. The compound of any one of embodiments 67-104, wherein R₉ and R₁₀ taken together with the atom to which they are attached form an oxo.
   114. The compound of any one of embodiments 67-113, wherein R₁₂ is O⁻, —OH, —O-alkyl, —O— haloalkyl, —O-cycloalkyl, or -OM.
   115. The compound of any one of embodiments 67-114, wherein R₁₂ is O⁻, —OH, —O-alkyl, —O— haloalkyl, or -OM.
   116. The compound of any one of embodiments 67-115, wherein R₁₂ is O—, —O-alkyl, —O— haloalkyl.
   117. The compound of any one of embodiments 67-115, wherein R₁₂ is —O-alkyl.
   118. The compound of any one of embodiments 113-117, wherein the —O-alkyl is —O—C₁₋₅alkyl.
   119. The compound of any one of embodiments 113-118, wherein the —O-alkyl is —OMe, —OEt, —OiPr, or -OtBu.
   120. The compound of any one of embodiments 67-115, wherein R₁₂ is —O-haloalkyl.
   121. The compound of any one of embodiments 114-116 and 120, wherein the —O-haloalkyl is —O—C₁₋₅haloalkyl.
   122. The compound of any one of embodiments 114-116 and 120, wherein the —O-haloalkyl is —OCH₂CF₃.
   123. The compound of any one of embodiments 67-115, wherein M is Na⁺, K⁺, or NH₄⁺.
   124. The compound of any one of embodiments 67-123, wherein R₁₄ is H, F, Cl, Br, or C₁₋₅alkyl.
   125. The compound of any one of embodiments 67-124, wherein R₁₄ is H.
   126. The compound of any one of embodiments 67-125, having the structure:

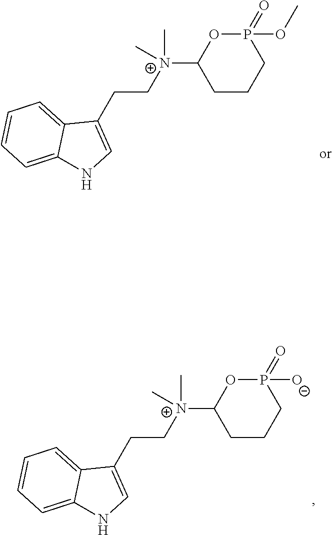 [SMILES: COP1(=O)CCCC([N+](C)(C)CCc2c[nH]c3ccccc23)O1 ; C[N+](C)(CCc1c[nH]c2ccccc12)C1CCCP(=O)([O-])O1]

or a pharmaceutically acceptable salt thereof.
127. The compound of any one of embodiments 1-66, wherein the compound of Formula (I) excludes the compounds described in International Application No. PCT/US2022/032918.
128. The compound of any one of embodiments 67-126, wherein the compound of Formula (II) excludes the compounds described in International Application No. PCT/US2022/032918.
129. A compound of Formula (I-A):

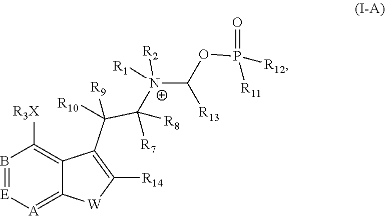

or a pharmaceutically acceptable salt thereof,
wherein:

A is N or C—VR₁₆;

B is N or C—YR₄;

E is N or C—ZR₅, wherein no more than two of A, B, and E are N;

W is O, S, or NR₆;

V, X, Y, and Z are each independently absent, O, S, or NH;

R₁ and R₂ are each independently H, D, deuterated alkyl, optionally substituted alkyl, optionally substituted cycloalkyl, or optionally substituted heteroalkyl;

R₃, R₄, R₅, and R₁₆ are each independently H, D, optionally substituted alkyl, optionally substituted cycloalkyl, optionally substituted heteroalkyl, or —P(O)(OH)₂;

R₆ is H, optionally substituted alkyl, optionally substituted cycloalkyl, alkylene-optionally substituted cycloalkyl, or optionally substituted heteroalkyl;

R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;

R₉ and R₁₀ are each independently H, D, halogen, optionally substituted alkyl, optionally substituted cycloalkyl, or optionally substituted heteroalkyl, or R₉ and R₁₀ taken together with the atom to which they are attached form an oxo;

R₁₁ and R₁₂ are each independently alkyl, deuterated alkyl, —O—, —OH, —OD, —O-alkyl, —O-cycloalkyl, —O-alkylene-cycloalkyl, —O-aryl, or —OM, wherein M is a pharmaceutically acceptable cation, or R₁₁ and R₁₂ together with the atoms to which they are attached form a heterocyclyl, and the alkyl, cycloalkyl, aryl and heterocyclyl are each optionally substituted;

R₁₃ is H, D, halogen, deuterated alkyl, optionally substituted alkyl, optionally substituted alkoxy, optionally substituted heteroalkyl, optionally substituted aryl, optionally substituted heteroaryl, alkylene-optionally substituted aryl, —C(O)-(alkylene- optionally substituted aryl), or -G-C(O)—NHR₁₅, or R₁₁ and R₁₃ taken together with the atoms to which they are attached form an optionally substituted heterocyclic ring comprising 4-7 carbon atoms;

R₁₄ is H, D, halogen, haloalkyl, —OH, an optionally substituted alkyl, optionally substituted cycloalkyl, or optionally substituted heteroalkyl; and

R₁₅ is H, optionally substituted alkyl, optionally substituted cycloalkyl, optionally substituted aryl, or optionally substituted heteroaryl, wherein the compound is not:

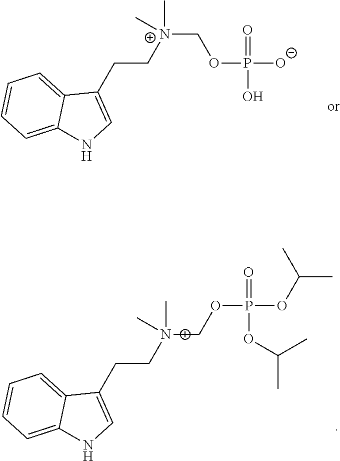 [SMILES: C[N+](C)(CCc1c[nH]c2ccccc12)COP(=O)([O-])O ; CC(C)OP(=O)(OC[N+](C)(C)CCc1c[nH]c2ccccc12)OC(C)C]

### EXAMPLES

Compounds of the present disclosure can be synthesized using the following exemplary methods or other methods that are known to those skilled in the art.

General reaction conditions are provided, and reaction products can be purified by known methods including silica gel chromatography using various organic solvents such as hexane, dichloromethane, ethyl acetate, methanol and the like or preparative reverse phase high pressure liquid chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

### Example 1. Synthesis of Compounds of the Disclosure

Compounds 1-10 of Table 5 were prepared as described by the methods below.

TABLE 5
Prepared Compounds 1-10 of the Disclosure.

| Compound | | |
|---|---|---|
| ID | Structure | Name |
| 1 | 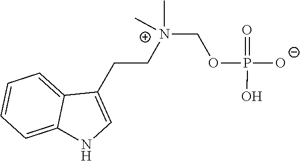 [SMILES: C[N+](C)(CCc1c[nH]c2ccccc12)COP(=O)([O-])O] | ((2-(1H-indol-3-yl)ethyl)dimethyl-λ⁴- azaneyl)methyl hydrogen phosphate |
| 2 | 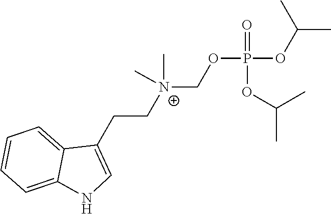 [SMILES: CC(C)OP(=O)(OC[N+](C)(C)CCc1c[nH]c2ccccc12)OC(C)C] | ((2-(1H-indol-3-yl)ethyl)dimethyl-λ⁴- azaneyl)methyl diisopropyl phosphate |
| 3 | 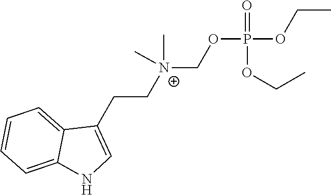 [SMILES: CCOP(=O)(OCC)OC[N+](C)(C)CCc1c[nH]c2ccccc12] | ((2-(1H-indol-3-yl)ethyl)dimethyl-λ⁴- azaneyl)methyl diethyl phosphate |
| 4 | 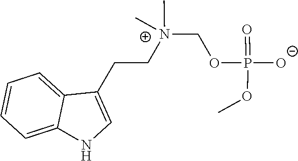 [SMILES: COP(=O)([O-])OC[N+](C)(C)CCc1c[nH]c2ccccc12] | ((2-(1H-indol-3-yl)ethyl)dimethyl-λ⁴- azaneyl)methyl phosphate |
| 5 | 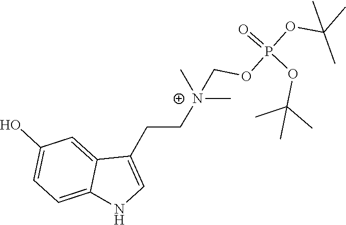 [SMILES: CC(C)(C)OP(=O)(OC[N+](C)(C)CCc1c[nH]c2ccc(O)cc12)OC(C)(C)C] | di-tert-butyl (((2-(5-hydroxy-1H-indol- 3-yl)ethyl)dimethyl-λ⁴-azaneyl)methyl) phosphate |
| 6 | 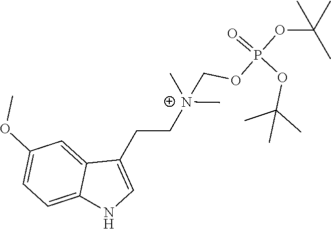 [SMILES: COc1ccc2[nH]cc(CC[N+](C)(C)COP(=O)(OC(C)(C)C)OC(C)(C)C)c2c1] | di-tert-butyl (((2-(5-methoxy-1H-indol- 3-yl)ethyl)dimethyl-λ⁴-azaneyl)methyl) phosphate |
| 7 | 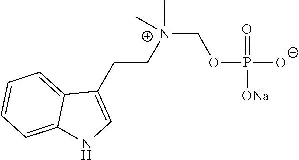 | monosodium mono(((2-(1H-indol-3- yl)ethyl)dimethyl-λ⁴-azaneyl)methyl phosphate) |
| 8 | 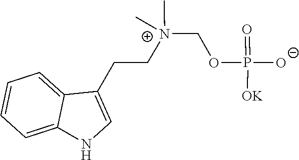 | potassium ((2-(1H-indol-3- yl)ethyl)dimethylammonio)methyl phosphate |
| 9 | 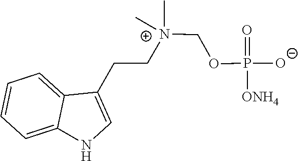 | ((2-(1H-indol-3- yl)ethyl)dimethylammonio)methyl (λ⁵- azaneyl) phosphate |
| 10 | 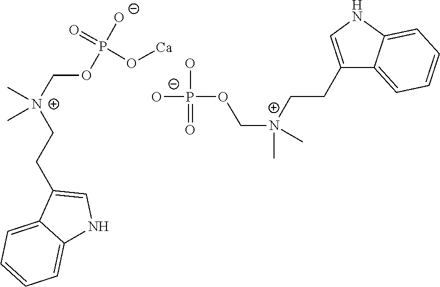 | calcium ((2-(1H-indol-3- yl)ethyl)dimethylammonio)methyl phosphate |

Compounds 1-4 were synthesized from a commercially available intermediate tryptophan according to Scheme 1.

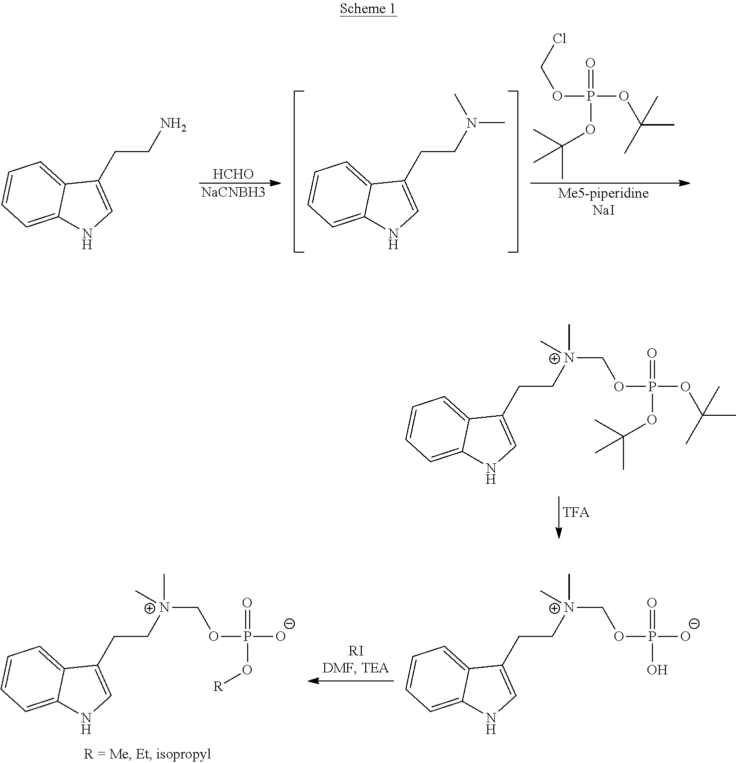

Compounds 5 and 6 were synthesized from commercially available intermediate 5-substituted tryptophan according to Scheme 2.

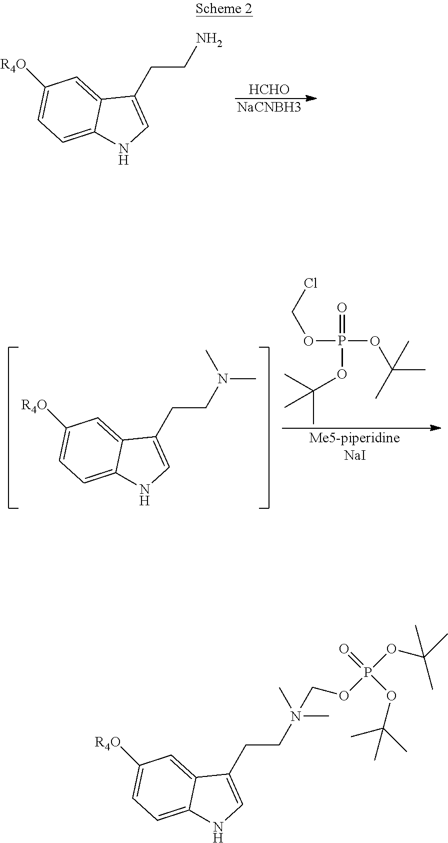

Salts of the disclosed compounds were synthesized in one step from compound 1 as shown in Scheme 3.

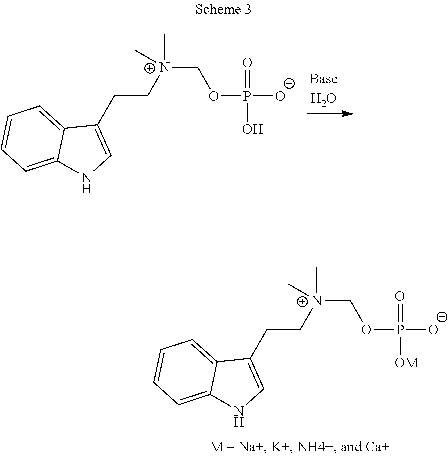

### Synthesis of Compound 1

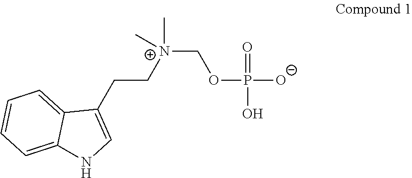 [SMILES: C[N+](C)(CCc1c[nH]c2ccccc12)COP(=O)([O-])O]

Step 1. To a solution of tryptamine (200 mg, 1.25 mmol, 1.0 equiv) in 3 mL of methanol was added sodium cyanoborohydride (197 mg, 3.13 mmol, 2.5 equiv), acetic acid (0.15 mL) and formaldehyde (254 mg, 3.13 mol, 2.5 equiv, 37% aq. solution) in methanol (1.5 mL) at 0° C. The mixture solution was stirred overnight at 25° C. and then concentrated under vacuum. The resulting solution was diluted with 20 mL of saturated sodium bicarbonate and extracted with ethyl acetate (3×20 mL). The combined organic layers were washed with brine (2×20 mL) of, dried over anhydrous sodium sulfate and concentrated under vacuum. The resulting mixture was dissolved in 2 mL of acetonitrile, then di-tert-butyl (chloromethyl) phosphate (274 mg, 1.06 mmol, 2.0 equiv), Me₅-piperidine (164.5 mg, 1.06 mmol, 2.0 equiv) and sodium iodide (8.0 mg, 0.05 mmol, 0.1 equiv) was added at 25° C. under nitrogen atmosphere. The mixture was stirred for 24 h at 40° C. The resulting solution was diluted with 20 mL of ethyl acetate and washed sequentially with saturated sodium bicarbonate solution (2×10 mL) and saturated sodium chloride solution (10 mL). The combined organic layers were washed with brine (2×20 mL), dried over anhydrous sodium sulfate, and concentrated under vacuum. The crude product (120 mg) was used in the next step directly without further purification.

Step 2. To a solution of intermediate (120 mg, 0.29 mmol, 1.0 equiv) from above Step 1 in dichloromethane (1.2 mL) under argon was added trifluoroacetic acid (0.6 mL) slowly at RT. The resulting solution was stirred for 1 h and then concentrated under reduced pressure. The crude product was purified by reverse phase-chromatography: Column, C₁₈ silica gel; mobile layer, water, and methanol (20% to 100% methanol over 15 min and hold 100% for 5 min); Detector, 220 nm. The fractions were concentrated under reduced pressure. Compound 1 was obtained as a white solid (28 mg, 28%). MS m/z [M+H]⁺ (ESI): 299.05. ¹H NMR (300 MHz, DMSO-d₆) δ 11.11 (s, 1H), 7.65 (d, J=7.8 Hz, 1H), 7.37 (d, J=8.1 Hz, 1H), 7.26 (s, 1H), 7.11-6.98 (m, 2H), 4.89 (d, J=8.7 Hz, 2H), 3.51-3.45 (m, 2H), 3.18-3.13 (m, 2H), 3.07 (s, 6H). ³¹P NMR (121 MHz, DMSO-d₆) δ−1.88.

### Synthesis of Compound 2

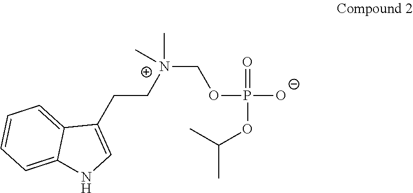 [SMILES: CC(C)OP(=O)([O-])OC[N+](C)(C)CCc1c[nH]c2ccccc12]

To a solution of Compound 1 (100 mg, 0.34 mmol, 1.00 equiv) in N,N-Dimethylformamide (1 mL) was added 2-Iodopropane (171 mg, 1.00 mmol, 3.0 equiv) and trimethylamine (101 mg, 1.00 mmol, 3.0 equiv) at 25° C. The resulting mixture was stirred overnight at 50° C. and then diluted with 20 mL of dichloromethane. The organic layer was washed with saturated aqueous sodium chloride solution (2×20 mL) and dried over anhydrous sodium sulfate. The resulting solution was concentrated under reduced pressure and purified by reverse-phase: Column, C18 silica gel; mobile phase, water (with 0.05% NH₄CO₃), 10% to 100% gradient in 20 min; detector, UV 254 nm. Compound 2 was obtained as a white solid (20 mg, 17%) of: MS m/z [M+H]⁺ (ESI): 341.05. ¹H NMR (400 MHz, DMSO-d₆) δ 10.98 (s, 1H), 7.65 (d, J=8.0 Hz, 1H), 7.36 (d, J=8.0 Hz, 1H), 7.26 (d, J=2.0 Hz, 1H), 7.12-7.08 (m, 1H), 7.03-6.99 (m, 1H), 4.87 (d, J=8.8 Hz, 2H), 4.29-4.22 (m, 1H), 3.50-3.45 (m, 2H), 3.19-3.15 (m, 2H), 3.08 (s, 6H), 1.12 (d, J=6.0 Hz, 6H).

### Synthesis of Compound 3

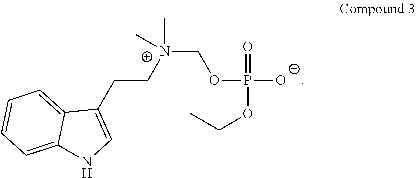 [SMILES: CCOP(=O)([O-])OC[N+](C)(C)CCc1c[nH]c2ccccc12]

In a similar manner described for Compound 2, Compound 3 was prepared from 100 mg of Compound 1 and 156 mg of iodoethane Compound 3 was obtained as a white solid (19 mg 17%). MS m/z [M+H]⁺ (ESI): 327.05. ¹H NMR (400 MHz, DMSO-d₆) δ 10.98 (s, 1H), 7.65 (d, J=7.6 Hz, 1H), 7.36 (d, J=8.0 Hz, 1H), 7.26-7.00 (m, 3H), 4.88 (d, J=8.8 Hz, 2H), 3.77-3.69 (m, 2H), 3.50-3.44 (m, 2H), 3.20-3.15 (m, 2H), 3.08 (s, 6H), 1.13-1.04 (m, 3H).

### Synthesis of Compound 4

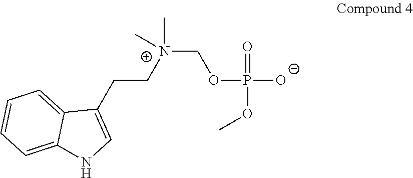 [SMILES: COP(=O)([O-])OC[N+](C)(C)CCc1c[nH]c2ccccc12]

In a similar manner described for Compound 2, Compound 4 was prepared from 100 mg of Compound 1 and 156 mg of iodomethane. The reaction was purified by Preparative-HPLC: Column: YMC Triart C18 ExRS, 20*150 mm, 5 μm; Mobile Phase A: 10 mmol NH₄HCO₃+0.05% NH₃·H₂O, Mobile Phase B: ACN; Flow rate: 20 mL/min; Gradient: 10% B to 34% B in 12 min, 34% B; Wavelength: 220 nm. Compound 4 was obtained as a white solid (19 mg). MS m/z [M+H]⁺ (ESI): 313.10. ¹H NMR (300 MHz, DMSO-d₆) δ 11.00 (s, 1H), 7.65 (d, J=7.8 Hz, 1H), 7.37 (d, J=7.8 Hz, 1H), 7.26 (d, J=2.1 Hz, 1H), 7.10-7.02 (m, 2H), 4.89 (d, J=8.4 Hz, 2H), 3.52 (s, 3H), 3.42-3.40 (m, 2H), 3.20-3.09 (m, 8H).

### Synthesis of Compound 5

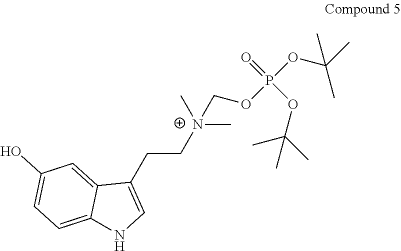 [SMILES: CC(C)(C)OP(=O)(OC[N+](C)(C)CCc1c[nH]c2ccc(O)cc12)OC(C)(C)C]

To a solution of 5-hydroxytryptamine (1 g, 5.2 mmol, 1.0 equiv) in methanol (10 mL) was added sodium cyanoborohydride (0.83 g, 13 mmol, 2.5 equiv), acetic acid (0.5 mL) and formaldehyde (1.1 mL, 13 mol, 2.5 equiv, 40% in water) at 0° C. The mixture solution was stirred overnight at 25° C. and then concentrated under vacuum. The crude product was dissolved in 2 mL of acetonitrile, then di-tert-butyl (chloromethyl) phosphate (480 mg, 1.9 mmol, 2.0 equiv), Me₅-piperidine (330 mg, 1.9 mmol, 2.0 equiv) and sodium iodide (15 mg, 0.2 mmol, 0.1 equiv) was added at 25° C. under nitrogen. The mixture was stirred for 24 h at 40° C. The resulting solution was diluted with ethyl acetate (20 mL) and washed with saturated sodium bicarbonate solution (2×10 mL) and saturated sodium chloride solution (10 mL), respectively. The combined organic layers were washed with brine (2×20 mL) and dried over anhydrous sodium sulfate. The resulting solution was concentrated under reduced pressure and purified by Preparative-HPLC: Column: XBridge Prep Phenyl OBD Column, 19*250 mm, 5 μm; Mobile Phase A: 10 mmol NH₄HCO₃+0.05% NH₃—H₂O, Mobile Phase B: ACN; Flow rate: 60 mL/min; Gradient: 20% B to 50% B in 8 min, 50% B; Wavelength: 254 nm. Compound 5 was obtained as a white solid (38 mg). MS m/z [M+H]⁺ (ESI): 371.15. ¹H NMR (300 MHz, DMSO-d₆) δ 10.79 (s, 1H), 9.35 (s, 1H), 7.16-7.09 (m, 3H), 6.64-6.60 (m, 1H), 4.92 (d, J=8.1 Hz, 2H), 3.50-3.48 (m, 2H), 3.07-3.02 (m, 8H), 1.33 (s, 9H).

### Synthesis of Compound 6

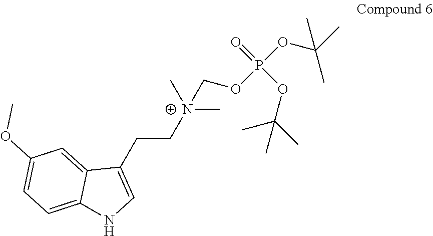 [SMILES: COc1ccc2[nH]cc(CC[N+](C)(C)COP(=O)(OC(C)(C)C)OC(C)(C)C)c2c1]

To a solution of 5-Methoxytryptamine (1 g, 5.2 mmol, 1.0 equiv) in methanol (10 mL) was added sodium cyanoborohydride (0.83 g, 13 mmol, 2.5 equiv), acetic acid (0.5 mL) and formaldehyde (1.1 mL, 13 mol, 2.5 equiv, 40% in water) at 0° C. The mixture solution was stirred overnight at 25° C. and then concentrated under vacuum. The crude product was dissolved in 2 mL of acetonitrile, then di-tert-butyl (chloromethyl) phosphate (480 mg, 1.9 mmol, 2.0 equiv), Me₅-piperidine (330 mg, 1.9 mmol, 2.0 equiv) and sodium iodide (15 mg, 0.2 mmol, 0.1 equiv) was added at 25° C. under nitrogen atmosphere. The mixture was stirred for 24 h at 40° C. The resulting solution was diluted with ethyl acetate (20 mL) and washed with saturated sodium bicarbonate solution (2×10 mL) and saturated sodium chloride solution (10 mL), respectively. The combined organic layers were washed with brine (2×20 mL) and dried over anhydrous sodium sulfate. The resulting solution was concentrated under reduced pressure and purified by Preparative-HPLC: Column: XBridge Prep OBD C₁₈ Column, 30*150 mm, 5 μm; Mobile Phase A: Water (10 mmol/L NH₄HCO₃+0.1% NH₃—H₂O), Mobile Phase B: ACN; Flow rate: 60 mL/min; Gradient: 5% B to 35% B in 9 min, 35% B; Wavelength: 220 nm. Compound 6 was obtained as a white solid (31 mg). MS m/z [M+H]⁺ (ESI): 385.20. ¹H NMR (400 MHz, DMSO-d₆) δ 10.88 (s, 1H), 7.27-7.15 (m, 3H), 6.75-6.72 (m, 1H), 4.86 (d, J=8.7 Hz, 2H), 3.79 (s, 3H), 3.48-3.43 (m, 2H), 3.15-3.10 (m, 2H), 3.08 (s, 6H), 1.31 (s, 9H).

### Synthesis of Compound 7

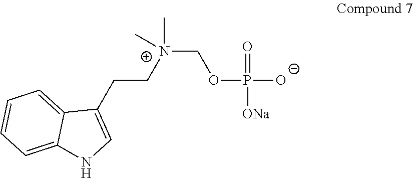

To a solution of Compound 1 (40 mg, 0.13 mmol, 1.00 equiv) in water (1 mL) were added sodium hydroxide (5.3 mg, 0.13 mmol, 1.0 equiv) at 25° C. The mixture was stirred for 15 min at 25° C. and the resulting solution was concentrated by lyophilization.) of Compound 7 was obtained as a white solid (30.0 mg, 70%). MS m/z [M+H]⁺ (ESI): 299.05. ¹H NMR (400 MHz, Deuterium Oxide) δ 7.57-7.52 (m, 1H), 7.43-7.39 (m, 1H), 7.18-7.13 (m, 2H), 7.11-7.06 (m, 1H), 4.66 (d, J=6.4 Hz, 2H), 3.38-3.31 (m, 2H), 3.07-3.01 (m, 2H), 2.92 (s, 6H).

### Synthesis of Compound 8

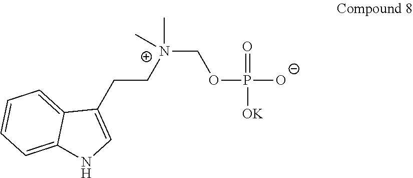

To a solution of Compound 1 (40 mg, 0.13 mmol, 1.0 equiv) in water (1 mL) were added potassium hydroxide (7 mg, 0.13 mmol, 1.0 equiv) at 25° C. The mixture was stirred for 15 min at 25° C. and the resulting solution was concentrated by lyophilization. Compound 8 was obtained as a white solid (32 mg, 71%). MS m/z [M+H]⁺ (ESI): 299.05. ¹H NMR (400 MHz, Deuterium Oxide) δ 7.57-7.52 (m, 1H), 7.43-7.39 (m, 1H), 7.18-7.13 (m, 2H), 7.11-7.06 (m, 1H), 4.66 (d, J=6.4 Hz, 2H), 3.38-3.31 (m, 2H), 3.07-3.01 (m, 2H), 2.92 (s, 6H).

### Synthesis of Compound 9

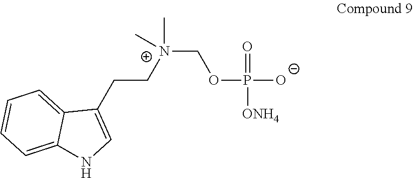

To a solution of Compound 1 (40 mg, 0.13 mmol, 1.00 equiv) in water (1 mL) were added ammonium hydroxide (23 mg, 0.13 mmol, 1.0 equiv) at 25° C. The mixture was stirred for 15 min at 25° C. and the resulting solution was concentrated by lyophilization. Compound 9 was obtained as a yellow solid (28 mg, 70%). MS m/z [M+H]⁺ (ESI): 299.05. ¹H NMR (400 MHz, Methanol-d₄) δ 7.69-7.61 (m, 1H), 7.39-7.34 (m, 1H), 7.24 (s, 1H), 7.16-7.04 (m, 2H), 4.92-4.90 (m, 2H), 3.67-3.57 (m, 2H), 3.32-3.27 (m, 2H), 3.17 (s, 6H).

### Synthesis of Compound 10

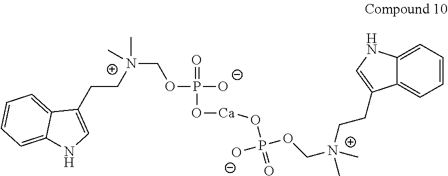

To a solution of Compound 1 (40 mg, 0.13 mmol, 1.00 equiv) in water (1 mL) were added calcium hydroxide (5 mg, 0.07 mmol, 0.5 equiv) at 25° C. The mixture was stirred for 15 min at 25° C. and the resulting solution was concentrated by lyophilization. Compound 10 was obtained as a white solid (33 mg, 72%). MS m/z [M+H]⁺ (ESI): 299.05. ¹H NMR (300 MHz, Deuterium Oxide) δ 7.52-7.47 (m, 1H), 7.38-7.33 (m, 1H), 7.15-7.00 (m, 3H), 4.62-4.61 (m, 2H), 3.36-3.25 (m, 2H), 3.06-2.94 (m, 2H), 2.88 (s, 6H).

TABLE 6
Prepared Compounds 11-28 of the Disclosure.

| Compound | | |
|---|---|---|
| ID | Structure | Name |
| 11 | 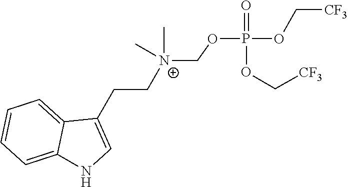 [SMILES: C[N+](C)(CCc1c[nH]c2ccccc12)COP(=O)(OCC(F)(F)F)OCC(F)(F)F] | ((2-(1H-indol-3-yl)ethyl)dimethyl-λ⁴- azaneyl)((bis(2,2,2- trifluoroethoxy)phosphoryl)oxy)methylium |
| 12 | 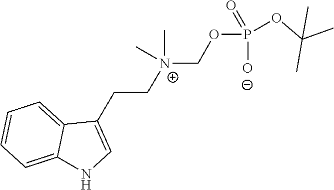 [SMILES: CC(C)(C)OP(=O)([O-])OC[N+](C)(C)CCc1c[nH]c2ccccc12] | ((2-(1H-indol-3- yl)ethyl)dimethylammonio)methyl tert- butyl phosphate |
| 13 | 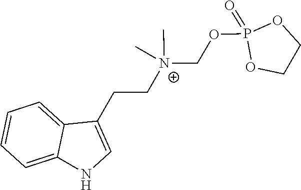 [SMILES: C[N+](C)(CCc1c[nH]c2ccccc12)COP1(=O)OCCO1] | 2-(1H-indol-3-yl)-N,N-dimethyl-N-(((2- oxido-1,3,2-dioxaphospholan-2- yl)oxy)methyl)ethan-1-aminium |
| 14 | 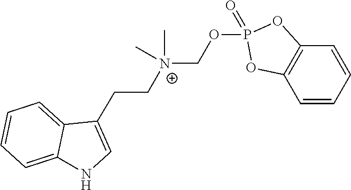 [SMILES: C[N+](C)(CCc1c[nH]c2ccccc12)COP1(=O)Oc2ccccc2O1] | 2-(1H-indol-3-yl)-N,N-dimethyl-N-(((2- oxidobenzo[d][1,3,2]dioxaphosphol-2- yl)oxy)methyl)ethan-1-aminium |
| 15 | 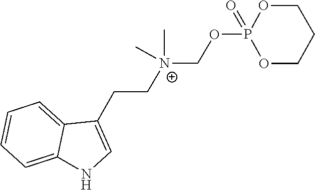 [SMILES: C[N+](C)(CCc1c[nH]c2ccccc12)COP1(=O)OCCCO1] | 2-(1H-indol-3-yl)-N,N-dimethyl-N-(((2- oxido-1,3,2-dioxaphosphinan-2- yl)oxy)methyl)ethan-1-aminium |
| 16 | 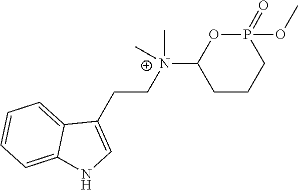 [SMILES: COP1(=O)CCCC([N+](C)(C)CCc2c[nH]c3ccccc23)O1] | N-(2-(1H-indol-3-yl)ethyl)-2-methoxy- N,N-dimethyl-1,2-oxaphosphinan-6- aminium 2-oxide |
| 17 | 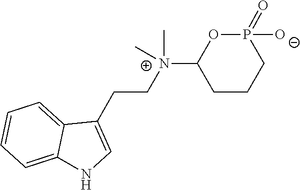 [SMILES: C[N+](C)(CCc1c[nH]c2ccccc12)C1CCCP(=O)([O-])O1] | 6-((2-(1H-indol-3- yl)ethyl)dimethylammonio)-1,2- oxaphosphinan-2-olate 2-oxide |
| 18 | 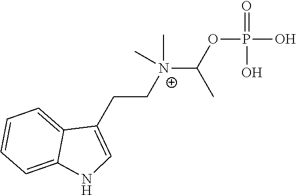 [SMILES: CC(OP(=O)(O)O)[N+](C)(C)CCc1c[nH]c2ccccc12] | 1-((2-(1H-indol-3-yl)ethyl)dimethyl-λ⁴- azaneyl)-1-(phosphonooxy)ethan-1-ylium |
| 19 | 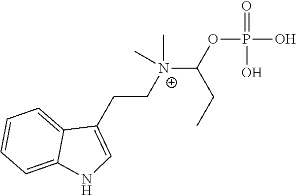 [SMILES: CCC(OP(=O)(O)O)[N+](C)(C)CCc1c[nH]c2ccccc12] | N-(2-(1H-indol-3-yl)ethyl)-N,N-dimethyl- 1-(phosphonooxy)propan-1-aminium |
| 20 | 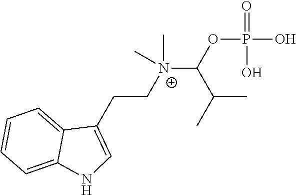 [SMILES: CC(C)C(OP(=O)(O)O)[N+](C)(C)CCc1c[nH]c2ccccc12] | 1-((2-(1H-indol-3-yl)ethyl)dimethyl-λ⁴- azaneyl)-2-methyl-1- (phosphonooxy)propan-1-ylium |
| 21 | 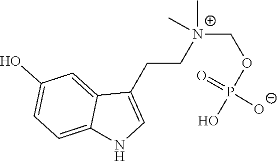 [SMILES: C[N+](C)(CCc1c[nH]c2ccc(O)cc12)COP(=O)([O-])O] | ((2-(5-hydroxy-1H-indol-3- yl)ethyl)dimethylammonio)methyl hydrogen phosphate |
| 22 | 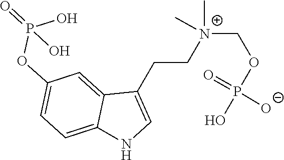 [SMILES: C[N+](C)(CCc1c[nH]c2ccc(OP(=O)(O)O)cc12)COP(=O)([O-])O] | (dimethyl(2-(5-(phosphonooxy)-1H-indol- 3-yl)ethyl)ammonio)methyl hydrogen phosphate |
| 23 | 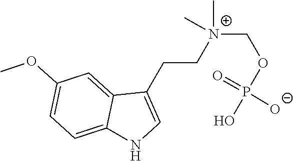 [SMILES: COc1ccc2[nH]cc(CC[N+](C)(C)COP(=O)([O-])O)c2c1] | ((2-(5-methoxy-1H-indol-3- yl)ethyl)dimethylammonio)methyl hydrogen phosphate |
| 24 | 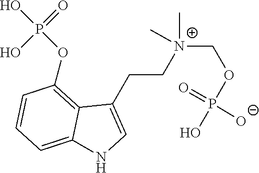 [SMILES: C[N+](C)(CCc1c[nH]c2cccc(OP(=O)(O)O)c12)COP(=O)([O-])O] | (dimethyl(2-(4-(phosphonooxy)-1H-indol- 3-yl)ethyl)ammonio)methyl hydrogen phosphate |
| 25 | 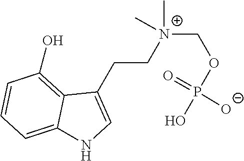 [SMILES: C[N+](C)(CCc1c[nH]c2cccc(O)c12)COP(=O)([O-])O] | ((2-(4-hydroxy-1H-indol-3- yl)ethyl)dimethyl-λ⁴- azaneyl)metheyliumyl hydrogen phosphate |
| 26 | 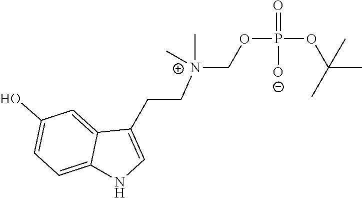 [SMILES: CC(C)(C)OP(=O)([O-])OC[N+](C)(C)CCc1c[nH]c2ccc(O)cc12] | tert-butyl (((2-(5-hydroxy-1H-indol-3- yl)ethyl)dimethylammonio)methyl) phosphate |
| 27 | 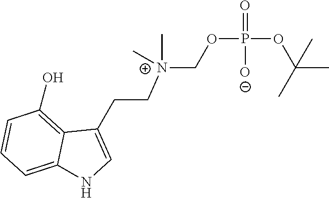 [SMILES: CC(C)(C)OP(=O)([O-])OC[N+](C)(C)CCc1c[nH]c2cccc(O)c12] | tert-butyl (((2-(4-hydroxy-1H-indol-3- yl)ethyl)dimethylammonio)methyl) phosphate |
| 28 | 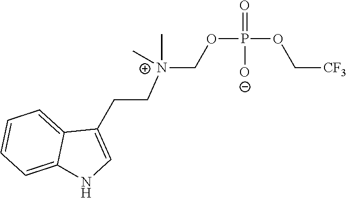 [SMILES: C[N+](C)(CCc1c[nH]c2ccccc12)COP(=O)([O-])OCC(F)(F)F] | ((2-(1H-indol-3- yl)ethyl)dimethylammonio)methyl (2,2,2- trifluoroethyl) phosphate |

### Example 2. Evaluation of Chemical Stability of Disclosed Compounds

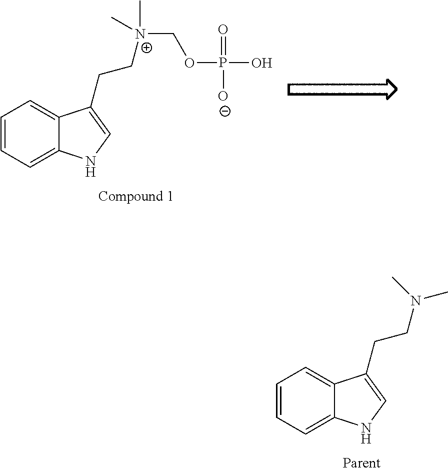 [SMILES: C[N+](C)(CCc1c[nH]c2ccccc12)COP(=O)([O-])O ; CN(C)CCc1c[nH]c2ccccc12]

### Illustration of the Prodrug Strategy:

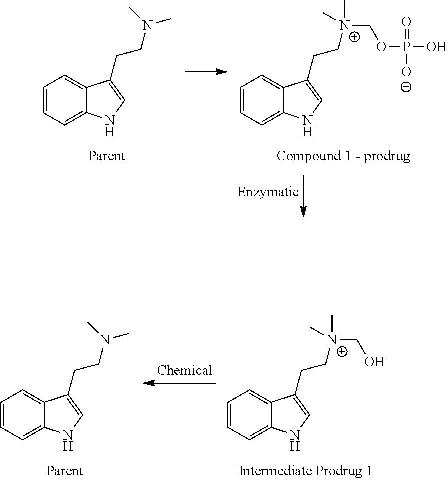 [SMILES: CN(C)CCc1c[nH]c2ccccc12 ; C[N+](C)(CCc1c[nH]c2ccccc12)COP(=O)([O-])O ; CN(C)CCc1c[nH]c2ccccc12 ; C[N+](C)(CO)CCc1c[nH]c2ccccc12]

Compound 1 was synthesized as part of a novel prodrug strategy for improving the water solubility of tertiary amine-containing drugs. As shown in the above scheme, tertiary amine DMT (parent) was converted in two-step reaction to the polar Intermediate Prodrug 1.

Intermediate Prodrug 1 releases the parent DMT in vivo through a two-step bio-reversion process. The rate determining first step is via an enzymatic process, where prodrug bio-reversion involves a phosphatase-catalyzed dephosphorylation to give the resultant hydroxymethyl quaternary ammonium intermediate and inorganic phosphate. The second step involves conversion of the hydroxymethyl quaternary ammonium intermediate chemically to DMT and formaldehyde at physiological pH.

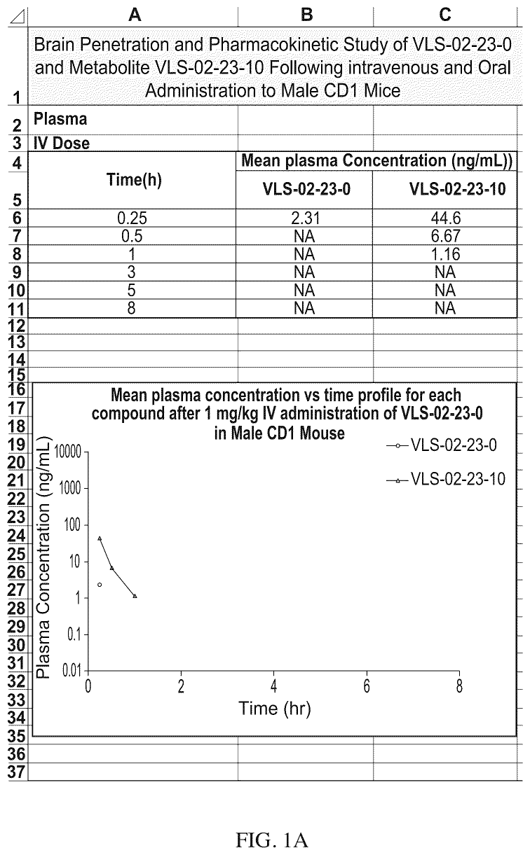

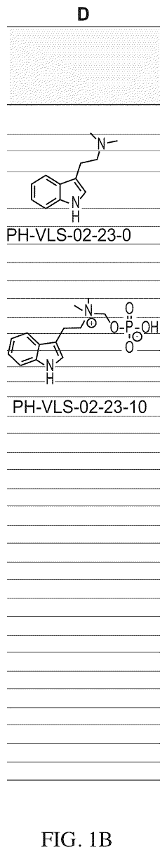

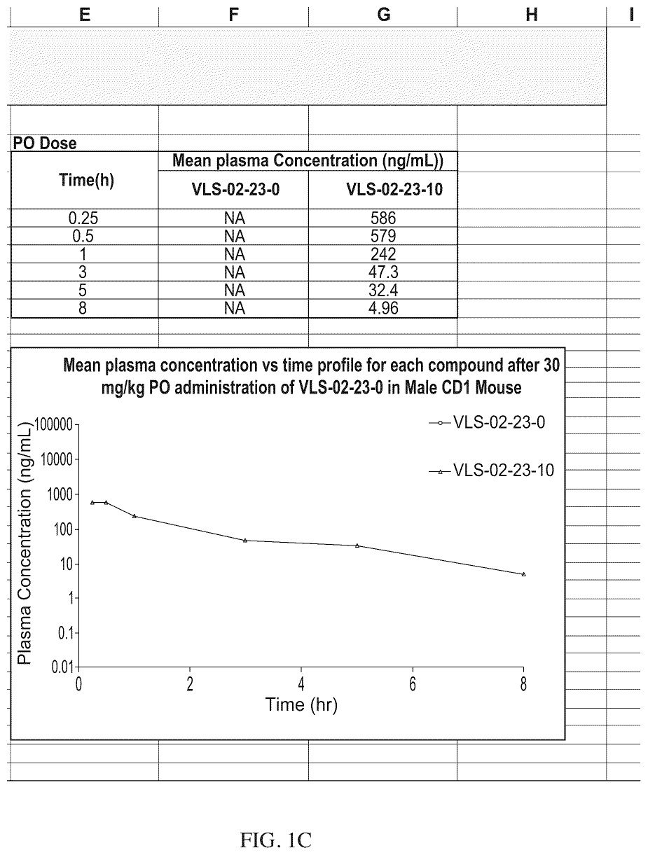

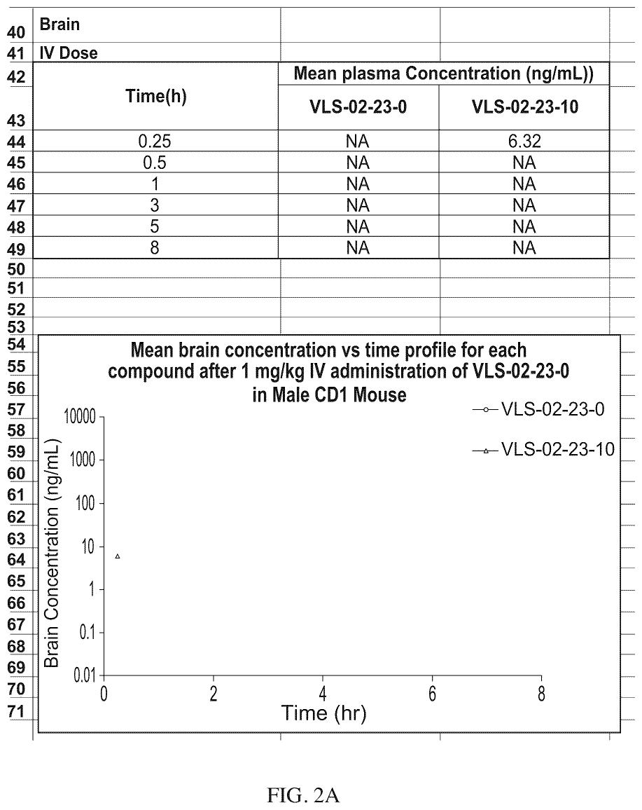

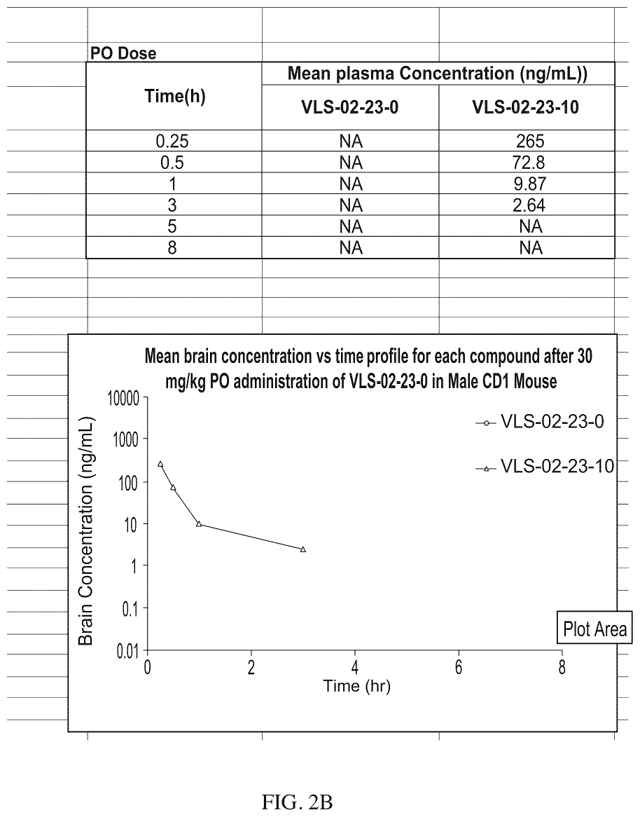

Chemical and plasma stability of prodrug Compound 1 was evaluated and the results enumerated in Table 7 and Table 8, respectively. PK studies of prodrug Compound 1 and metabolite (VLS-02-23-10) in mice were also conducted and both plasma exposure and brain exposure were analyzed as shown in FIGS. 1A-1C and FIGS. 2A-2B, respectively.

### Chemical Stability of N-Phosphonooxymethyl Prodrug Compound 1

TABLE 7
Stability Results of Test Compounds in PBS pH 6.5, SGF with and without Pepsin

| | | T½ | Remaining Percentage (%) | | | | |
|---|---|---|---|---|---|---|---|
| Compound | Incubation | (min) | 0 min | 30 min | 60 min | 120 min | 240 min |
| Chlorambucil | PBS (pH 6.5) | 43.17 | 100.00 | 61.12 | 43.17 | 15.12 | 2.15 |
| Erythromycin | SGF with | 40.26 | 100.00 | 56.55 | 34.88 | 11.22 | 1.60 |
| | pepsin | | | | | | |
| Erythromycin | SGF without | 37.36 | 100.00 | 59.45 | 33.60 | 10.27 | 1.20 |
| | pepsin | | | | | | |
| Compound 1 | PBS (pH 6.5) | 9999.00 | 100.00 | 104.71 | 106.37 | 107.29 | 117.35 |
| Compound 1 | SGF with | 6227.35 | 100.00 | 96.70 | 106.43 | 102.36 | 96.82 |
| | pepsin | | | | | | |
| Compound 1 | SGF without | 9999.00 | 100.00 | 98.76 | 107.08 | 101.90 | 108.80 |
| | pepsin | | | | | | |

### Plasma Stability of N-Phosphonooxymethyl Prodrug Compound 1

TABLE 8
Stability Results of Test Compounds in Mouse Plasma

| | | T½ | Remaining Percentage (%) | | | | |
|---|---|---|---|---|---|---|---|
| Compound | Species | (min) | 0 min | 15 min | 30 min | 60 min | 120 min |
| Propantheline | Mouse | 34.09 | 100.00 | 80.81 | 64.04 | 34.86 | 9.05 |
| Compound 1 | | 568.34 | 100.00 | 114.24 | 110.21 | 109.95 | 91.16 |

1. A compound of Formula (I-A): 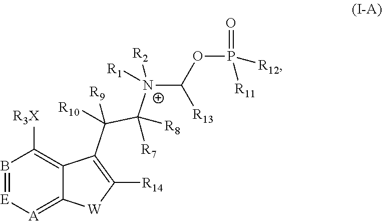
or a pharmaceutically acceptable salt thereof,
wherein:
A is N or C—VR₁₆;
B is N or C—YR₄;
E is N or C—ZR₅, wherein no more than two of A, B, and E are N;
W is O, S, or NR₆;
V, X, Y, and Z are each independently absent, O, S, or NH;
R₁ and R₂ are each independently H, D, deuterated alkyl, optionally substituted alkyl, optionally substituted cycloalkyl, or optionally substituted heteroalkyl;
R₃, R₄, R₅, and R₁₆ are each independently H, D, optionally substituted alkyl, optionally substituted cycloalkyl, optionally substituted heteroalkyl, or —P(O)(OH)₂;
R₆ is H, optionally substituted alkyl, optionally substituted cycloalkyl, alkylene- optionally substituted cycloalkyl, or optionally substituted heteroalkyl;
R₇ and R₈ are each independently H, D, halogen, optionally substituted alkyl, optionally substituted cycloalkyl, or optionally substituted heteroalkyl, or R₇ and R₈ taken together with the atom to which they are attached form an optionally substituted cycloalkyl ring;
R₉ and R₁₀ are each independently H, D, halogen, optionally substituted alkyl, optionally substituted cycloalkyl, or optionally substituted heteroalkyl, or R₉ and R₁₀ taken together with the atom to which they are attached form an oxo;
R₁₁ and R₁₂ are each independently alkyl, deuterated alkyl, —O⁻, —OH, —OD, —O-alkyl, —O— cycloalkyl, —O-alkylene-cycloalkyl, —O-aryl, or —OM, wherein M is a pharmaceutically acceptable cation, or R₁₁ and R₁₂ together with the atoms to which they are attached form a heterocyclyl, and the alkyl, cycloalkyl, aryl and heterocyclyl are each optionally substituted;
R₁₃ is H, D, halogen, deuterated alkyl, optionally substituted alkyl, optionally substituted alkoxy, optionally substituted heteroalkyl, optionally substituted aryl, optionally substituted heteroaryl, alkylene-optionally substituted aryl, —C(O)-(alkylene- optionally substituted aryl), or —O—C(O)—NHR₁₅, or R₁₁ and R₁₃ taken together with the atoms to which they are attached form an optionally substituted heterocyclic ring comprising 4-7 carbon atoms;
R₁₄ is H, D, halogen, haloalkyl, —OH, an optionally substituted alkyl, optionally substituted cycloalkyl, or optionally substituted heteroalkyl; and
R₅ is H, optionally substituted alkyl, optionally substituted cycloalkyl, optionally substituted aryl, or optionally substituted heteroaryl.

2. The compound of claim 1, wherein A is C—VR₁₆.

3. The compound of claim 1, wherein V is absent.

4. The compound of claim 2, wherein R₁₆ is H.

5. The compound of claim 1, wherein B in C—YR₄.

6. The compound of claim 5, wherein Y is absent.

7. The compound of claim 5, wherein Y is G.

8. The compound of claim 5, wherein R₄ is H, optionally substituted C₁₋₅alkyl, or —P(O)(OH)₂.

9. The compound of claim 5, wherein R₄ is methyl.

10. The compound of claim 1, wherein E is C—ZR₅.

11. The compound of claim 10, wherein Z is absent.

12. The compound of claim 10, wherein R₅ is H.

13. The compound of claim 1, wherein X is absent.

14. The compound of claim 1, wherein X is O.

15. The compound of claim 1, wherein R₃ is H, optionally substituted C₁₋₅alkyl, or —P(O)(OH)₂.

16. The compound of claim 1, wherein W is NR₆.

17. The compound of claim 16, wherein R₆ is H or optionally substituted C₁₋₅alkyl.

18. The compound of claim 16, wherein R₆ is H.

19. The compound of claim 1, wherein R₁ and R₂ are each independently H or optionally substituted C₁₋₅alkyl.

20. The compound of claim 1, wherein R₁ and R₂ are each optionally substituted C₁₋₅alkyl.

21. The compound of claim 20, wherein R₁ and R₂ are methyl.

22. The compound of claim 1, wherein R₇ and R₈ are each independently H, F, or optionally substituted C₁₋₅alkyl.

23. The compound of claim 1, wherein R₇ and R₈ are each H.

24. The compound of claim 1, wherein R₉ and R₁₀ are each independently H, F, or optionally substituted C₁₋₅alkyl.

25. The compound of claim 1, wherein R₉ and R₁₀ are each H.

26. The compound of claim 1, wherein R₉ and R₁₀ taken together with the atom to which they are attached form an oxo.

27. The compound of claim 1, wherein R₁₁ and R₁₂ are each independently —O—, —OH, —O—C₁₋₅alkyl, or -OM.

28. The compound of claim 1, wherein R₁₁ is O⁻ and R₁₂ is —OH, —O—C₁₋₅alkyl, or -OM.

29. The compound of claim 1, wherein R₁₁ and R₁₂ are each —O—C₁₋₅alkyl.

30. The compound of claim 27, wherein R₁₁ and R₁₂ are each independently —OMe, —OEt, —OiPr, or -OtBu.

31. The compound of claim 27, wherein R₁₁ and R₁₂ are each independently —O—C₁₋₅haloalkyl.

32. The compound of claim 27, wherein R₁₁ and R₁₂ are —OCH₂CF₃.

33. The compound of claim 1, wherein M is Na⁺, K⁺, or NH₄⁺.

34. The compound of claim 1, wherein R₁₁ and R₁₂ are each —OH.

35. The compound of claim 1, wherein R₁₁ and R₁₂ taken together with the atoms to which they are attached form an optionally substituted heterocyclyl.

36. The compound of claim 1, wherein R₁₁ and R₁₂ taken together with the atoms to which they are attached form an optionally substituted 5- to 12-membered heterocyclyl.

37. The compound of claim 35, wherein the optionally substituted heterocyclyl is:

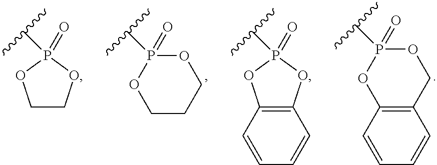 [SMILES: *P1(=O)OCCO1 ; *P1(=O)OCCCO1 ; *P1(=O)Oc2ccccc2O1 ; *P1(=O)OCc2ccccc2O1]

38. The compound of claim 1, wherein R₁₁ and R₁₂ are each deuterated alkyl.

39. The compound of claim 1, wherein R₁₁ and R₁₂ are each deuterated methyl.

40. The compound of claim 1, wherein R₁₃ is H or optionally substituted C₁₋₅alkyl.

41. The compound of 40, wherein R₁₃ is methyl, ethyl, or isopropyl.

42. The compound of claim 1, wherein R₁₄ is H, F, Cl, Br, or C₁₋₅alkyl.

43. The compound of claim 1, wherein R₁₄ is H.

44. The compound of claim 1, wherein R₁₄ is haloalkyl.

45. The compound of claim 1, wherein R₁₄ is —CF₃.

46. The compound of claim 1, having the structure: 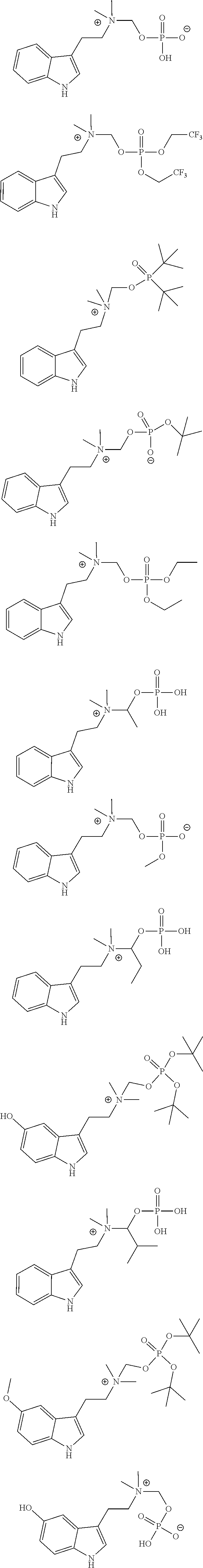 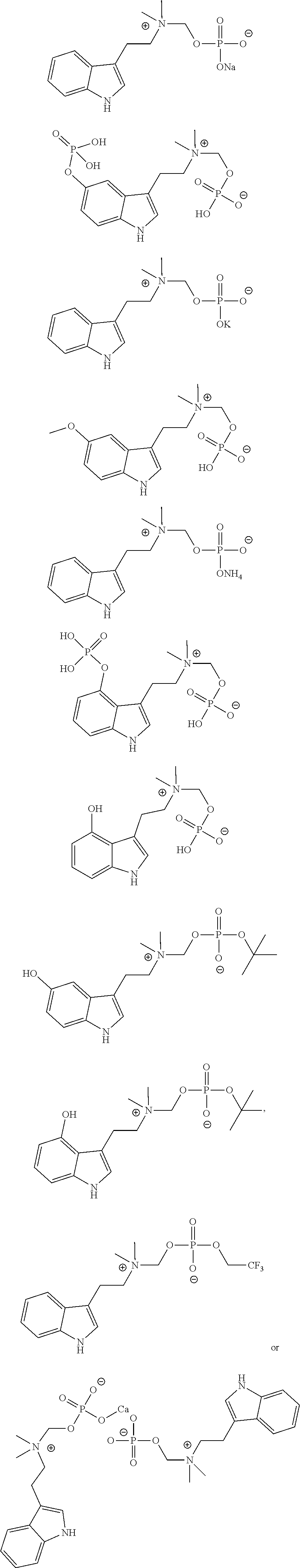
or a pharmaceutically acceptable salt thereof.

47. The compound of claim 1, having the structure: 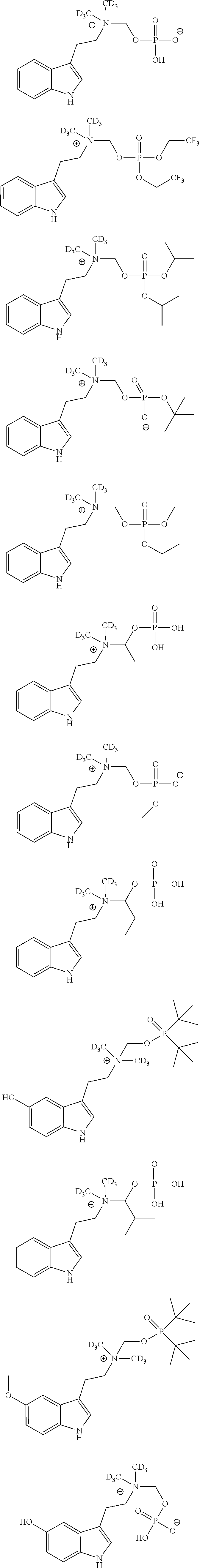 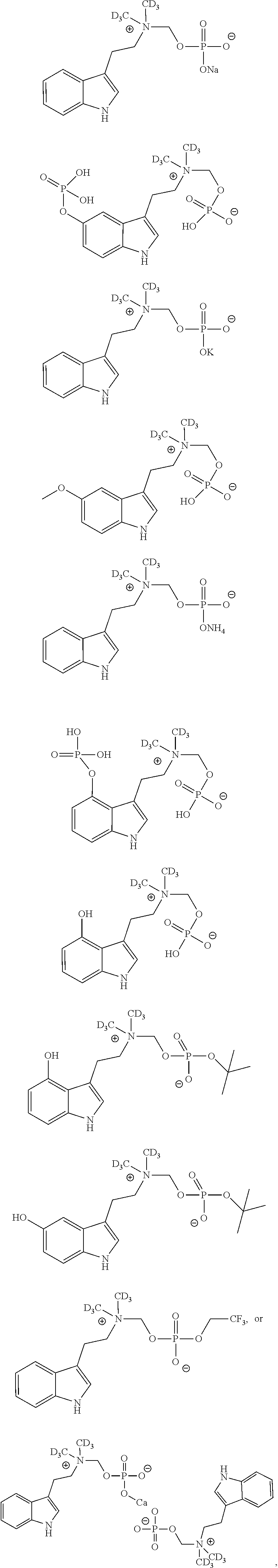
or a pharmaceutically acceptable salt thereof.

48. The compound of claim 1, having the structure: 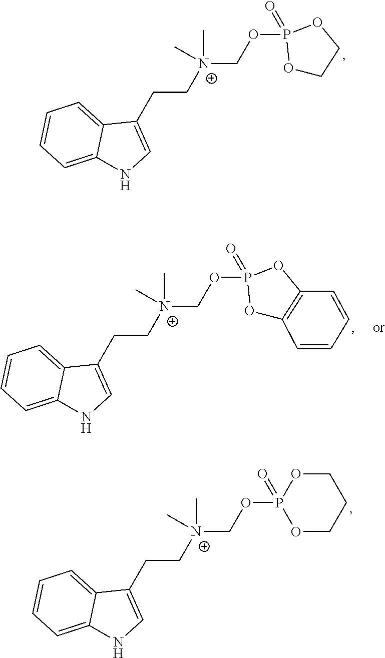 [SMILES: C[N+](C)(CCc1c[nH]c2ccccc12)COP1(=O)OCCO1 ; C[N+](C)(CCc1c[nH]c2ccccc12)COP1(=O)Oc2ccccc2O1 ; C[N+](C)(CCc1c[nH]c2ccccc12)COP1(=O)OCCCO1]
or a pharmaceutically acceptable salt thereof.

49. The compound of claim 1, having the structure: 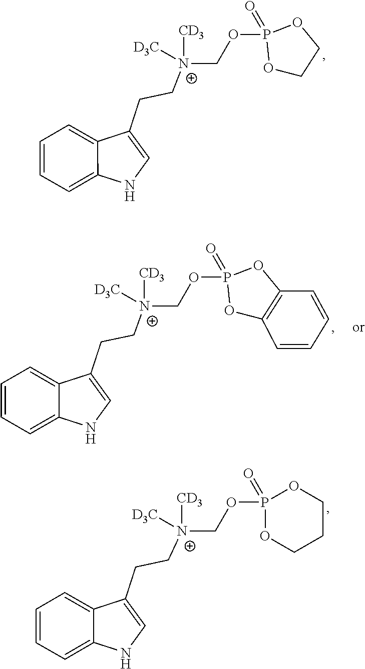 [SMILES: [2H]C([2H])([2H])[N+](CCc1c[nH]c2ccccc12)(COP1(=O)OCCO1)C([2H])([2H])[2H] ; [2H]C([2H])([2H])[N+](CCc1c[nH]c2ccccc12)(COP1(=O)Oc2ccccc2O1)C([2H])([2H])[2H] ; [2H]C([2H])([2H])[N+](CCc1c[nH]c2ccccc12)(COP1(=O)OCCCO1)C([2H])([2H])[2H]]
or a pharmaceutically acceptable salt thereof.

50-77. (canceled)

78. The compound of claim 1, having the structure: 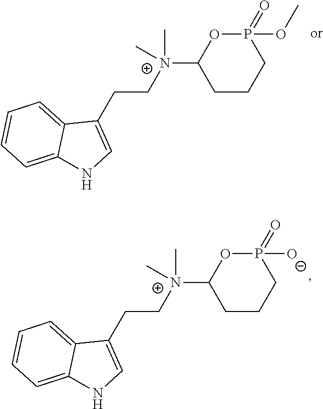 [SMILES: COP1(=O)CCCC([N+](C)(C)CCc2c[nH]c3ccccc23)O1 ; C[N+](C)(CCc1c[nH]c2ccccc12)C1CCCP(=O)([O-])O1]
or a pharmaceutically acceptable salt thereof.

79. The compound of claim 1, having the structure: 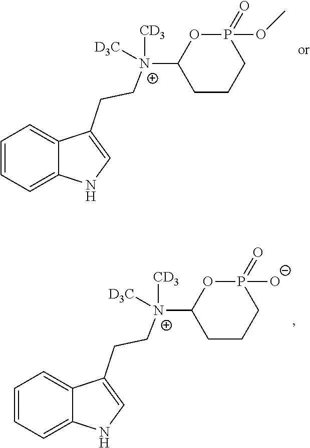 [SMILES: [2H]C([2H])([2H])[N+](CCc1c[nH]c2ccccc12)(C1CCCP(=O)(OC)O1)C([2H])([2H])[2H] ; [2H]C([2H])([2H])[N+](CCc1c[nH]c2ccccc12)(C1CCCP(=O)([O-])O1)C([2H])([2H])[2H]]
or a pharmaceutically acceptable salt thereof.

80. A composition comprising (i) a compound of claim 1 or a pharmaceutically acceptable salt thereof, and (ii) a pharmaceutically acceptable excipient.

81. An oral dosage form comprising the pharmaceutical composition of claim 80.

82. A method of treating post-traumatic stress disorder (PTSD) in a subject in need thereof, comprising administering a pharmaceutical composition of 80 to the subject.

83. A method of treating depression in a subject in need thereof, comprising administering a pharmaceutical composition of claim 80 to the subject.

84. The method of claim 83, wherein the depression is major depressive disorder (MDD) or treatment-resistant depression (TRD).

85. A method of treating an anxiety disorder in a subject in need thereof, comprising a pharmaceutical composition of claim 80 to the subject.
